(19) United States
(12) Patent Application Publication
(10) Pub. No.: US 2019/0298717 A1
(43) Pub. Date: Oct. 3, 2019

(54) Hydralazine and Active Derivatives Thereof for Neuronal Cell Survival and Regeneration

(71) Applicant: BOARD OF REGENTS, THE UNIVERSITY OF TEXAS SYSTEM, Austin, TX (US)

(72) Inventors: Hamid Mirzaei, Dallas, TX (US); Asish Ray Chaudhuri, Helotes, TX (US); Sivaramakrishna Yadavalli, Irving, TX (US); Esmail Dehghan, Dallas, TX (US); Zhang Yiqiang, Dallas, TX (US); Tambar K. Uttam, Dallas, TX (US); Indumathy S. Subramaniyan, Coppell, TX (US); Maryam Dehghani, Dallas, TX (US); Bin Xu, Dallas, TX (US); Bahar Saremi, Arlington, TX (US)

(21) Appl. No.: 16/085,368

(22) PCT Filed: Mar. 14, 2017
(86) PCT No.: PCT/US2017/022340

§ 371 Date: Sep. 14, 2018

Related U.S. Application Data
(60) Provisional application No. 62/307,816, filed on Mar. 14, 2016.

(51) Int. Cl. A61K 31/502; A61P 25/28

(52) U.S. Cl. CPC A61K 31/502; A61P 25/28

(57) ABSTRACT

The present invention includes a method of protecting or treating neural cells from degeneration comprising: identifying a neural cell in need of protection from at least one of radical oxidative stress, increased mitochondrial biogenesis, decreased intracellular protein aggregation or neurofibrillary tangles, increase of at least one of cellular NAD or ATP levels, activation of autophagy, removal of protein aggregates, prevents GSK3β or Tau protein phosphorylation, or increase neuronal plasticity; and providing the neural cell with a therapeutically effective amount of a hydralazine or active bioequivalent thereof sufficient to reduce radical oxidative stress, increase mitochondrial biogenesis, decrease intracellular protein aggregation or neurofibrillary tangles, increase of at least one of cellular NAD or ATP levels, activate autophagy, remove protein aggregates, prevent GSK3β or Tau protein phosphorylation, or increase neuronal plasticity, thereby protecting the neural cells from degeneration.

### TECHNICAL FIELD OF THE INVENTION

The present invention relates in general to the field of degenerative disease of the nervous systems, and more particularly, to composition and method of using hydralazine and active derivatives thereof for neuronal cell survival and regeneration.

### BACKGROUND OF THE INVENTION

Without limiting the scope of the invention, its background is described in connection with treatments for degenerative disease of the nervous system.

United States Patent Publication No. 2006/0160848, filed by Burcham et al., is entitled “Method of controlling damage mediated by alpha, beta-unsaturated aldehydes”, and is said to relate to a method for inhibiting the reaction of an alpha, beta-unsaturated aldehyde with a biological molecule, the method including the step of administering hydralazine and/or dihydralazine in an amount that is effective to reduce the rate of reaction of the alpha, beta-unsaturated aldehyde with the biological molecule.

United States Patent Publication No. 2009/0215687, filed by Ratan et al., is entitled “Compounds for Enhancing Hypoxia Inducible Factor Activity and Methods of Use” and is said to relate to methods for enhancing Hypoxia inducible factor-1 (HIF) activity in a cell by contacting the cell with any one of the following compounds: 3,6-bis[2-(dimethylamino)ethoxy]-9h-xanthen-9-onedihydrochloride, 2,8-bis[dimethylaminoacetyl]dibenzofurin dihydrochloride hydrate, tilorone analogue R-9536-DA, indoprofen, ciclopiroxolamine, tryptophan, ansindione, nabumetone, oxybendazole, albendazole, tropicamide, pramoxine hydrochloride, atenolol, mebendazole, carbetapentane citrate, monensin sodium, methoxyvone, hydroxyzine, phenazopyridine, clofoctol, ipraflavone, zomepirac, biochanin A, xylometazoline hydrochloride, fenbendazole, pirenzepine, triprolidine hydrochloride, daidzein, tripelennamine citrate, colchicines, aminopyridine, trimethoprim, helenine, hydroxyurea, amiodarone hydrochloride, clindamycin hydrochloride, sulfachlorpyridazine, mephenesin, semustine, clofivric acid, clofibrate, ibuprofen, hyoscyamime, nafcillin sodium, piperin, clidinium bromide, trioxsalen, hydralazine and HIF alpha protein fused to a carrier peptide.

United States Patent Publication No. 2013/0116215, filed by Coma et al., entitled “Combination Therapies For Treating Neurological Disorders” is said to relate to novel pharmaceutical combinations useful for the treatment of neurological diseases, specifically neurodegenerative diseases. The pharmaceutical combinations are said to demonstrate additive or synergistic effect in silico and in vivo, and are also said to relate to methods of treatment of neurological and neurodegenerative diseases including the pharmaceutical combinations of a calcium channel blocker and one bisphosphonate.

### SUMMARY OF THE INVENTION

In one embodiment, the present invention includes a method of protecting neural cells from degeneration or treating degenerated neural cells comprising: identifying a neural cell in need of protection or treatment from at least one of: radical oxidative stress, increased mitochondrial biogenesis, decreased intracellular protein aggregation or neurofibrillary tangles, decreased cellular NAD or ATP levels, activation of autophagy, removal of protein aggregates, inhibition of GSK3β or Tau protein phosphorylation, or increased neuronal plasticity or dendrite formation; and providing the neural cell with a therapeutically effective amount of a hydralazine or active bioequivalent thereof sufficient to protect the neural cells from degeneration or treat the neurodegeneration of the neural cells. In one aspect, the step of reducing radical oxidative stress comprises activating Nrf2 signaling to neutralize and detoxify the neural cell cytoplasm from radical oxidative stress. In another aspect, the active bioequivalent of hydralazine is selected from at least one of 1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, Phthalazin-1 (2H)-one, 6-Hydrazinyl-2-methyl-[1,2,4]triazolo[5,1-a]phthalazine, Isonicotinohydrazide, or salts thereof. In another aspect, the hydralazine or active bioequivalent (1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, Phthalazin-1(2H)-one, 6-Hydrazinyl-2-methyl-[1,2,4]triazolo[5,1-a]phthalazine, Isonicotinohydrazide) thereof is adapted for oral, intravenous, intramuscular, alveolar, intranasal, peritoneal, subcutaneous, enteral, parenteral, rectal, or topical administration. In another aspect, the hydralazine or active bioequivalent thereof is provided in an amount that at least one of: activates deacetylase enzymes, activates SIRT1, activates SIRT5, increases mitochondrial biogenesis, restores mitochondrial function, or elevates cellular NAD or ATP levels. In another aspect, the hydralazine or active bioequivalent thereof is provided in an amount that activates autophagy to remove organelles or protein aggregates. In another aspect, the hydralazine or active bioequivalent thereof is provided in an amount that at least one of reduces the formation of neurofibrillary tangles (NFTs) by inhibiting the phosphorylation of GSK3β kinase or Tau protein. In another aspect, the hydralazine or active bioequivalent thereof is provided in an amount that increases neural plasticity or dendrite production. In another aspect, the hydralazine or active bioequivalent thereof is provided in an amount that increases the inactive phosphorylated form of pGSK33. In another aspect, the hydralazine or active bioequivalent thereof is provided in an amount that protects neuronal cells from toxicity induced by at least one of Aβ (1-42), amyloid, or NFT. In another aspect, the hydralazine or active bioequivalent thereof is provided in an amount that increases at least one of functional synaptic plasticity or dendrite formation. In another aspect, the hydralazine or active bioequivalent thereof is provided in an amount of 10, 20, 30, 40, 50, 60, 70, 80, 90, 100, 125, 150, 175, 200, 225, 250, 275 or 300 milligrams per day. In another aspect, the neural cell degeneration is not acrolein-mediated. In another aspect, the method further comprises the step of determining that the neural cell degeneration is not acrolein-mediated, and then providing the subject with the effective amount of hydralazine or active bioequivalent thereof. In another aspect, the subject is a mammal. In another aspect, the disease or condition is selected from at least one of a neurodegenerative disease or disorder selected from at least one of non-viral encephalopathy, Alzheimer's disease, Parkinson's disease, amyotrophic lateral sclerosis, Huntington's disease, a tauopathy, an age-related neurodegenerative disease or disorder, muscular sclerosis, a rare genetic neurodegenerative disease, a disease or disorder involving a microbial infection of the nervous system, poliomyelitis, a physical or ischemic injury of the nervous system, seizure, stroke, trauma, epilepsy, a disease or disorder involves the presence of a chemical neurotoxic agent and/or of an oxidative stress. In another aspect, the hydralazine or active bioequivalent thereof further comprises one or more pharmaceutically acceptable excipients.

Another embodiment of the present invention includes a method of treating a neurodegenerative disease or condition in a subject comprising the step of administering a therapeutically effective amount of at least one of Hydralazine, 1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, Phthalazin-1(2H)-one, 6-Hydrazinyl-2-methyl-[1,2,4]triazolo[5,1-a]phthalazine, Isonicotinohydrazide, or salts thereof. In one aspect, the disease or condition is selected from at least one of a neurodegenerative disease or disorder selected from at least one of non-viral encephalopathy, Alzheimer's disease, Parkinson's disease, amyotrophic lateral sclerosis, Huntington's disease, a tauopathy, an age-related neurodegenerative disease or disorder, muscular sclerosis, a rare genetic neurodegenerative disease, a disease or disorder involving a microbial infection of the nervous system, poliomyelitis, a physical or ischemic injury of the nervous system, seizure, stroke, trauma, epilepsy, a disease or disorder involves the presence of a chemical neurotoxic agent and/or of an oxidative stress. In another aspect, the hydralazine or active bioequivalent thereof is provided as a pharmaceutically-acceptable salt is selected from acetate, besylate (benzenesulfonate), benzoate, bicarbonate, bitartrate, bromide, calcium edentate, camphorsulfonate (camsylate), carbonate, chloride, chlorotheophyllinate, citrate, edetate, ethanedisulfonate (edisylate), ethanesulfonate (esylate), fumarate, gluceptate (glucoheptonate), gluconate, glucuronate, glutamate, hexylresorcinate, hydroxynaphthoate, hippurate, iodide, isethionate, lactate, lactobionate, lauryl sulfate (estolate), malate, maleate, mandelate, mesylate, methanesulfonate, methylnitrate, methylsulfate, mucate, naphthoate, napsylate, nitrate, octadecanoate, oleate, oxalate, pamoate, pantothenate, phosphate, polygalacturonate, salicylate, stearate, succinate, sulfate, sulfosalicylate, tannate, tartrate, teoclate, toluene sulfonate (tosylate), and trifluoroacetate. In another aspect, neurodegenerative disease or condition is not acrolein-mediated. In another aspect, method further comprises the step of determining that the neurodegenerative disease or condition is not acrolein-mediated, and then providing the subject with the effective amount of hydralazine or active bioequivalent thereof. In another aspect, subject is a human.

Yet another embodiment of the present invention includes a method of treating a subject suffering from a neurodegenerative disorder or condition comprising administering an effective amount of a hydralazine or active bioequivalent thereof, wherein the hydralazine or active bioequivalent thereof is effective to at least one of reduce radical oxidative stress, increase mitochondrial biogenesis, decrease intracellular protein aggregation or neurofibrillary tangles, increase cellular NAD and/or ATP levels, activate autophagy, remove protein aggregates, prevent GSK3β or Tau protein phosphorylation, or increase neuronal plasticity and/or dendrite formation.

Another embodiment of the present invention includes a method of identifying a hydralazine or active analog for preventing or treating a neurodegenerative disorder, the method comprising: a) measuring at least one of radical oxidative stress, increased mitochondrial biogenesis, decreased intracellular protein aggregation or neurofibrillary tangles, decreased cellular NAD or ATP levels, activation of autophagy, removal of protein aggregates, prevents GSK3β or Tau protein phosphorylation, or increase neuronal plasticity or dendrite formation from neural tissue or cells suspected of having a neurodegenerative disorder from a set of patients; b) administering a candidate drug to a first subset of the patients, and a placebo to neural tissue or cells from a second subset of the patients; c) repeating step a) after the administration of the candidate drug or the placebo; and d) determining if the candidate drug reduces the neurodegenerative disorder that is statistically significant as compared to any reduction occurring in the second subset of patients, wherein a statistically significant reduction indicates that the candidate drug is useful in treating said disease state.

Another embodiment of the present invention includes a method of reducing radical oxidative stress in a subject comprising the step of administering a therapeutically effective amount of a hydralazine or active bioequivalent thereof in an amount sufficient to increase mitochondrial biogenesis.

Another embodiment of the present invention includes a method of increasing mitochondrial biogenesis in a subject comprising the step of administering a therapeutically effective amount of a hydralazine or active bioequivalent thereof in an amount sufficient to increase mitochondrial biogenesis.

Another embodiment of the present invention includes a method of increasing cellular NAD and/or ATP levels in a subject comprising the step of administering a therapeutically effective amount of a hydralazine or active bioequivalent thereof in an amount sufficient to increase cellular NAD and/or ATP levels.

Another embodiment of the present invention includes a method of activating at least one of autophagy, removal of protein aggregates, or prevents GSK3β, or Tau protein phosphorylation, in a subject comprising the step of administering a therapeutically effective amount of a hydralazine or active bioequivalent thereof in an amount sufficient to activate autophagy, removal of protein aggregates, or prevents GSK3β, or Tau protein phosphorylation.

Another embodiment of the present invention includes a method of increase neuronal plasticity or dendrite formation in a subject comprising the step of administering a therapeutically effective amount of a hydralazine or active bioequivalent thereof in an amount sufficient to increase neuronal plasticity and/or dendrite formation.

Yet another embodiment of the present invention includes a composition for treating degenerated neural cells comprising a therapeutically effective amount of at least one of Hydralazine, 1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, Phthalazin-1 (2H)-one, 6-Hydrazinyl-2-methyl-[1,2,4]triazolo[5,1-a]phthalazine, Isonicotinohydrazide, or salts thereof, sufficient to reduce at least one of radical oxidative stress, increase mitochondrial biogenesis, decrease intracellular protein aggregation or neurofibrillary tangles, increases NAD and/or ATP levels, activate autophagy, remove protein aggregates, prevent GSK3β or Tau protein phosphorylation, or increase neuronal plasticity or dendrite formation, thereby protecting the neural cells from degeneration. In one aspect, the hydralazine or active bioequivalent thereof is adapted for oral, enteral, parenteral, intravenous, intramuscular, pulmonary, rectal, or subcutaneous administration. In another aspect, the hydralazine or active bioequivalent thereof further comprises one or more pharmaceutically acceptable excipients.

Another embodiment of the present invention includes a composition for preventing neural cells degeneration comprising a therapeutically effective amount of at least one of Hydralazine, 1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, Phthalazin-1 (2H)-one, 6-Hydrazinyl-2-methyl-[1,2,4]triazolo[5,1-a]phthalazine, Isonicotinohydrazide, or salts thereof, sufficient to reduce at least one of radical oxidative stress, increase mitochondrial biogenesis, decrease intracellular protein aggregation or neurofibrillary tangles, increases NAD and/or ATP levels, activate autophagy, remove protein aggregates, prevent GSK3β or Tau protein phosphorylation, or increase neuronal plasticity or dendrite formation, thereby protecting the neural cells from degeneration. In one aspect, the hydralazine or active bioequivalent thereof is adapted for oral, enteral, parenteral, intravenous, intramuscular, pulmonary, rectal, or subcutaneous administration. In another aspect, the hydralazine or active bioequivalent thereof further comprises one or more pharmaceutically acceptable excipients.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D show that Hydralazine protects SH-SY5Y from oxidative stress induced cell death. FIG. 1A shows protein carbonyls that were measured in SH-SY5Y cells treated with hydralazine, H₂O₂, or H₂O₂ and hydralazine using 2-DNPH assay. Hydralazine treatment significantly reduced the carbonyl level raised by H₂O₂. FIG. 1B shows Hydrazine, a compound with the same aldehyde chelating functional group as hydralazine, also reduced the free carbonyls in the lysate. FIG. 1C shows the viability of SH-SY5Y cells treated with hydralazine, H₂O₂, or H₂O₂ and hydralazine was measured. The viability of cells under H₂O₂ induced stress was significantly improved with hydralazine treatment in a dose-dependent manner. FIG. 1D shows that Hydrazine treatment did not rescue cells from H₂O₂ stress indicating that hydralazine mode of action does not depend on carbonyl sequestration. (n=3, t-test, *p<0.05 and **p<0.01).

FIGS. 2A1-2A-2, 2B and 2C show a global comparative proteomics screen identifies Nrf2 as a pathway activated with hydralazine treatment. FIG. 2A-1-2A-2 is the SILAC workflow for identification of pathways activated with hydralazine treatment (n=2). FIG. 2B shows the proteins and their ratios (treated/untreated) were submitted for IPA analysis where Nrf2 pathway was found activated. Raw MS data for proteins SQSTM1, FTH1, and GSTK1 are shown. FIG. 2C shows that Nrf2 pathway activation reported by IPA signified with Z score of 0.156 and P value of 1.97E-10.

FIGS. 3A to 3C show that Hydralazine enhances Nrf2 signaling in SH-SY5Y cells. FIG. 3A shows that Hydralazine inhibits the interaction between Nrf2 and Keap1. SH-SY5Y cells were treated with 0, 10, and 20 μM of hydralazine for 24 h before co-IP was performed. FIG. 3B shows that Nrf2 translocates to the nucleus with hydralazine treatment. Cells were treated same as (FIG. 3A) before subjected to cell fractionation and Western blotting analysis. FIG. 3C shows that Hydralazine treatment increases Nrf2 phosphorylation quantified using an antibody specific to Nrf2 phosphorylation at serine 40 in nuclear and cytosol fractions. In all experiments, 5 μM of sulforaphane (+) was used as a positive control for Nrf2 signaling. For all experiments: n=3 independent samples; t-test, *p<0.05, **p<0.01.

FIGS. 4A to 4F show that the activation of Nrf2 is functional. FIG. 4A shows that Trx1, a potent regulator of the Nrf2-Keap1 response system, is upregulated with hydralazine treatment. FIG. 4B shows the luciferase activity was measured as an indicator of the transcriptional activation of Nrf2 target genes. FIG. 4C shows the expression of other proteins regulated by Nrf2 increases with hydralazine treatment. FIG. 4D shows that the Hydralazine treatment increases the intracellular level of GSH in SH-SY5Y cells. FIG. 4E shows that cell viability measured for SH-SY5Y cells (Ctrl and Nrf2 KD) treated with different concentrations of H₂O₂. FIG. 4F shows that the Hydralazine cannot reduce cytotoxicity in Nrf2 KD cells. SH-SY5Y cells (Ctrl and Nrf2 KD) were treated with hydralazine (Hyd), H₂O₂, or hydralazine plus H₂O₂ (H₂O₂+Hyd) for 24 h (n=3 independent treatments, t-test, *p<0.05 and **p<0.01 between hydralazine-treated and control cells or as indicated on the graph). Cytotoxicity was measured using a commercial LDH activity detection kit.

FIGS. 5A to 5J show that the SKN-1 pathway is activated with hydralazine treatment in C. elegans. FIG. 5A shows the intestinal SKN-1::GFP fluorescence and corresponding DIC images of LG357 animals treated with hydralazine (Hyd) or vehicle (Ctrl) (X40). FIG. 5B shows quantification of percentage of worms with high (>15 GFP-positive intestinal nuclei), medium (5-15 GFP-positive intestinal nuclei), or low (<5 GFP-positive intestinal nuclei) nuclear SKN-1::GFP (n=100, three independent trials, t-test, **p<0.01). FIG. 5C shows intestinal nuclei GFP signal intensity quantified by image J. for the worms shown in part A (n=40, three independent trials, t-test,*p<0.01). FIG. 5D shows GFP signal intensity of SKN-1 was increased in ASI neurons of ldIs7 transgenic worms measured by fluorescence microscopy (X40). FIG. 5E shows ASI neurons GFP signal intensity quantified by image J. for the worms shown in part D (n=100, three independent trials, t-test, **p<0.01). FIG. 5F shows an immunoblot showing higher amounts of SKN-1 isoforms B and C in the transgenic ldIs7 animals treated by 0 or 100 μM hydralazine for 72 hours (validated with IP-WB). FIG. 5G shows GST-4p::GFP signal quantification in transgenic worms dvIs19 in different time points after treatment with 100 μm hydralazine (n=35, three independent trials, t-test, **p<0.01). FIG. 5H shows Hydralazine treatment (100 μM for 72 h) induces GST-4p::GFP expression in worms fed control RNAi plasmid but not in worms fed with skn-1(RNAi). FIG. 5I shows the level of superoxide (O₂⁻) decreases with hydralazine treatment in WT worms treated with hydralazine but not in SKN-1 mutant worms (Data are represented as mean±SD, n=3, t-test, *p<0.05). FIG. 5J shows a volcano plot showing activation of SKN-1/Nrf2 pathway in wild type C. elegans. Proteins were quantified in both treated and untreated animals using label-free mass spectrometry and ratios were uploaded for identification of activated pathways via IPA analysis. SKN-1/Nrf2 pathway was among the activated pathways with Z score of 3.317. A list of human orthologs of SKN-1 pathway members and their Log FC is shown in a table below the plot.

FIG. 6A to 6H show that Hydralazine activates Nrf2 pathway in cells under stress from tau and/or rotenone toxicity and improves their viability. FIG. 6A shows aggregate-positive cells grew slower than aggregate-negative cells due to tau aggregate cytotoxicity. Hydralazine improves the viability of aggregate-positive cells only. FIG. 6B shows the concentration of superoxide is higher in aggregate-positive cells and it decreases with hydralazine treatment in a dose-dependent manner in both aggregate-positive and -negative cells. Superoxide content of the cells was measured after 24 h of treatment using hydroethidine (HE) assay. FIG. 6C shows Nrf2 and HO-1 were quantified by Western blot in aggregate-negative and positive cells treated with 0, 10, and 20 μM of hydralazine. FIG. 6D shows Hydralazine does not improve the viability of AP Nrf2 KD cells. FIG. 6E shows the concentration of superoxide does not decrease in AP Nrf2 KD cells. FIG. 6F shows Hydralazine attenuates the reduction in Nrf2 expression resulting from rotenone treatment. FIG. 6G shows Hydralazine improves the viability of both aggregate-positive and negative cells that are under rotenone stress. FIG. 6H shows Hydralazine improves the viability of primary neuronal cells under rotenone stress in a dose-dependent manner. For the data, statistical significance was indicated on the graph between treatment and control group or as indicated by the connecting lines (n=3, t-test, *p<0.05 and **p<0.01).

FIGS. 7A to 71 show that Hydralazine attenuates rotenone toxicity and extends lifespan and healthspan in C. elegans. FIG. 7A shows Hydralazine significantly protects C. elegans against rotenone-induced mortality (n=117, Log-rank (Mantel-Cox) test, p<0.0001). FIG. 7B shows worms pretreated with hydralazine were protected from rotenone but not worms pretreated with hydrazine or NaCl. Data are represented as mean±SD, n=210, two independent trials, t-test, **p<0.01. FIG. 7C shows Hydralazine protection was significantly decreased when skn-1 was knocked down or mutated (i.e. skn-1(RNAi) or skn-1(zu135)). Mutant skn-1(zu67) with an inactive isoform C and functional isoform B showed significantly higher protection against rotenone toxicity. Integration of SKN-1 isoforms B and C resulted in maximum protection against rotenone-induced death (ldIs7). Data are represented as mean±SD, n=240, three independent trials, t-test, *p<0.05 and **p<0.01. FIG. 7D shows Hydralazine pretreatment prevented rotenone-induced reduction in locomotion in N2 worms. skn-1(zu135) mutants were more vulnerable to rotenone toxicity but not completely unprotected when treated with hydralazine. Data are represented as mean±SD, n=70, three independent trials, t-test, *p<0.05 and **p<0.01. FIG. 7E shows Hydralazine treatment increased C. elegans lifespan in a dose-dependent manner. Maximum extension was observed with 100 μm hydralazine treatment. FIG. 7F shows Hydralazine-mediated extension of lifespan is SKN-1 dependent. skn-1 knockdown blocks longevity benefits of hydralazine. FIG. 7G shows Hydralazine treatment did not extend lifespan in skn-1(zu135) mutant. Expression of skn-1 isoform b in skn-1(zu67), partially restored longevity effects of hydralazine. FIG. 7H shows Expression of skn-1 isoforms b by transgenes geIs9 partially restored longevity benefits of hydralazine, while expression of isoform c in transgenic geIs10 did not. FIG. 7I shows healthspans of two C. elegans populations (N2 and skn-1(zu135)) were evaluated by measuring locomotor performance in young (5 days for N2, 4 days for mutants), mid-age (10 days for N2, 8 days for mutants) and old (15 days for N2 and 12 days for mutants) worms. Data are represented as mean±SD, n=60, three independent trials, t-test, *p<0.05 and **p<0.01. Hydralazine prevents age-related decline in locomotion in N2 worms but not the skn-1(zu135). P values, number of trials and population sizes for lifespan studies are shown in Table 1.

FIGS. 8A to 8F show that Hydralazine activates SKN-1/Nrf2 pathway in worms treated with rotenone. FIG. 8A is a Volcano plot showing the ratio (hydralazine+rotenone/rotenone) distribution of proteins quantified by label-free mass spectrometry. SKN-1/Nrf2 pathway nodes are shown in red. FIG. 8A shows the proteins ratios obtained by label free mass spectrometry were uploaded for IPA analysis. SKN-1/Nrf2 was number four in the top five activated stress response pathways (p-value cutoff of 0.05). FIG. 8C is a Volcano plot showing the ratio (rotenone/Ctrl) distribution of proteins quantified by label-free mass spectrometry. SKN-1/Nrf2 pathway nodes are shown in red. FIG. 8D shows the results of IPA analysis for worms treated with rotenone compared to control. SKN-1/Nrf2 was not among activated pathways (p-value cutoff of 0.05). FIG. 8E is a Volcano plot showing the hydralazine+rotenone/Ctrl ratio distribution of proteins quantified by label-free mass spectrometry. SKN-1/Nrf2 pathway nodes are shown in red (p-value cutoff of 0.05). FIG. 8F shows that the SKN-1/Nrf2 pathway was among activated pathways when worms treated with rotenone and hydralazine were compared to control worms (p-value cutoff of 0.05).

FIGS. 9A and 9B show the rescue effect of hydralazine on doxorubicin induced cell toxicity and NAD depletion. FIG. 9A shows that hydralazine alleviates doxorubicin mediated cell toxicity and NAD depletion in a dose-dependent manner. SH-SY5Y cells were treated with doxorubicin (Dox) (0.2 μM) in the presence of different concentrations of hydralazine. Viability of the cells was measured by Cell Titer Glo after 24 and 48 hours of treatments. FIG. 9B shows that hydralazine increased NAD content of the cells. Cells were treated for 48 hours with different concentrations of hydralazine and 20 μM isoniazid (Iso) as negative control. Hydralazine also recovered doxorubicin (0.2 μM) induced NAD depletion of the cells. Values are presented as mean±SDE (*p<0.05, **p<0.01).

FIGS. 10A and 10B show that hydralazine treatment increases the expression of key enzymes involved in cellular bioenergetics. FIG. 10A shows that hydralazine up-regulates transcription of NAD biosynthetic enzyme; NMNAT-1 and NAD-dependent sirtuin enzymes; SIRT1 and SIRT5. Cells were treated with 20 μM hydralazine or isoniazid (referred as -) for 12 hours. mRNA relative expressions for these three enzymes were then measured by qRT-PCR. Data were normalized to actin. FIG. 10B shows a western blot analysis of NMNAT-1, SIRT1, SIRT5 and COX-IV as representatives of mitochondrial electron transfer chain enzymes. Cells were treated with two different doses of hydralazine (10 and 20 μM) or isoniazid (20 μM). The intensity of the bands was normalized to actin. Data are mean±SEM of triplicate measurements.

FIGS. 11A to 11E show that hydralazine induces mitochondrial biogenesis and improves mitochondrial function. FIG. 11A shows mitochondrial mass in SH-SY5Y cells treated with two different doses of hydralazine (10 and 20 μM), 20 μM isoniazid as negative control and 20 μM resveratrol as positive control. Mitochondrial mass was measured by staining the cells with Mito tracker deep red followed by flow cytometric analysis. FIG. 11B shows the mitochondrial DNA content of SH-SY5Y cells treated with 20 μM hydralazine measured by quantitative PCR. DNA copy number of mitochondria was calculated by amplification of ND5 or m-RNR, as mitochondrial genes, relative to LPL as a nucleus specific DNA sequence. Relative expression values were normalized to untreated cells.

FIG. 11C shows confocal microscopic images visualizing mitochondrial membrane potentials in SH-SY5Y untreated cells (CTR) and cells treated with 20 μM hydralazine (HYD), isoniazid (−) and resveratrol (+). FIG. 11D shows a flow cytometric analysis that confirms quantitative changes in mitochondrial membrane potential in cells treated with two different concentrations of hydralazine (10 and 20 μM) and 20 μM isoniazid or resveratrol as negative and positive controls. Cells were stained by TMRE to tag active mitochondria. Florescence signal was quantified by Flow Jo software. FIG. 11E shows the relative amount of ATP production in the SHY-SY5 cells treated with 10 or 20 μM hydralazine for 3 days. ATP levels were measured by bioluminescent detection of luciferase activity. All data are presented normalized to cell number.

FIGS. 12A to 12C show that hydralazine increases viability and corrects lower energy output in taupathy model cells. FIG. 12A compares the viability of cells expressing mutated tau; RD(P301L/V337M)-YFP (“LM”), as soluble proteins (clone 1) or insoluble aggregate (clone 9) with or without treatment with 10 μM hydralazine for 48h. FIG. 11B shows that while mutated tau aggregates lower the relative amounts of NAD in clone 9 cells, hydralazine treatment can reverse this phenotype and increase the NAD content in this taupathy model cells. FIG. 11C shows lower ATP level in cells under tau stress pointing at mitochondrial dysfunction, and an increase in ATP content in cells treated with hydralazine.

FIGS. 13A to 13D show the results of a series of studies conducted on SH-SHY5Y cell to evaluate hydralazine's cytotoxicity and determine its mode of action. FIG. 13A shows percent cell viability measured by luminometry using cell titer glow luminescence assay for cells treated with increasing concentrations of hydralazine (0.195-400 μM) for 48 hours. FIG. 13B shows the results of a series of cell viability assays on SH-SY5Y cells treated with hydrazine (12.5 and 25 μM) in the presence or absence MG132 (10 μM, proteasome inhibitor) or bafilomycin (20 nM, autophagy inhibitor) or both in unchallenged and challenged conditions with H₂O₂(100 μM) for 4 hours. Results are expressed as means±SD from three independent experiments. Asterisks and signs denote values that are significantly different from those obtained from control cells; * P<0.05, ** P<0.01, & P<0.001, and # P<0.0001. This data show that when proteasome is inhibited hydralazine can still rescue cells from H₂O₂ induced stress but when autophagy is inhibited, hydralazine can no longer rescue the cells from stress indicating that hydralazine mode of action is related to autophagy. FIG. 13C shows the effect of hydralazine on autophagic marker proteins LC3-I and II. SH-SY5Y cells were treated with 0, 5, and 10 μM of hydralazine in presence and absence of bafilomycin (20 nM, autophagy inhibitor) for 4 hours. Cell lysates were then subjected to Western blot analysis. Statistical significance was indicated by the star (*p<0.05 between control and hydralazine treated cells). FIG. 13D shows the effect of hydralazine on 26S proteasome activity. SH-SY5Y cells were treated with 0 or 5, 10, 20 μM of hydralazine for 4 hours and assayed for proteasome activity using 26S proteasome luminescence assay kit. No significant activation was observed in 4 hours.

FIGS. 14A and 14B show mRFP-GFP-LC3 fluorescence signal recorded after 24 or 48 hours treatment with hydralazine. FIG. 14A shows the mRFP-GFP-LC3 fluorescence signal recorded from knock in MEF cells treated with hydralazine (10 μM) or rapamycin (10 μM) for 24 and 48 hours. Cells were then subjected to confocal analysis and mRFP-GFP (yellow representing autophagosome) and mRFP (red puncta representing autophagolysosomes) were recorded; scale bar, 10 gtm. FIG. 14B shows each correlation plot is derived from the field shown in the immunofluorescence image. The co-localization of mRFP with GFP signal from tfLC3 puncta was measured using ImageJ software.

FIGS. 15A to 15D show the effect of hydralazine on clearance of aggregated mutant huntingtin protein (EGFP-HDQ74). FIG. 15A is a confocal analysis showing the clearance of aggregated EGFP-HDQ74 in stable inducible PC12 cells expressing EGFPHDQ74. Transgene expression was induced with doxycycline for 8 hours, and then switched off (by removing doxycycline). Cells were treated with hydralazine or untreated (control) for 48 hours. The fraction of EGFP-positive cells expressing aggregates under different conditions were plotted as odds ratios taking Ctrl value as 1. Error bars show 95% confidence interval. FIG. 15B is a confocal analysis of aggregated EGFP-HDQ74 clearance in presence of 5 mM 3-MA (autophagy inhibitors) and 20 nM bafilomycin (autophagolysosomal flux inhibitor) with hydralazine treatment. The fraction of EGFP-positive cells with aggregates were plotted as odds ratios taking Ctrl value as 1. Error bars show 95% confidence interval. FIG. 15C shows a Western blot analysis of soluble EGFP-HDQ74 aggregate clearance in stable inducible PC12 cells expressing mutant EGFP-HDQ74. Cells were treated with hydralazine. Rapamycin (10M) was used as positive control. Right panel is densitometry analysis of EGFP-HDQ74 level normalized to actin. Error bars show SD. FIG. 15D is a confocal analysis of aggregated EGFP-HDQ74 clearance in presence or absence of autophagy protein 5 (ATG5) shRNA with hydralazine treatment. The fraction of EGFP-positive cells expressing aggregates were plotted as odds ratios taking Ctrl value as 1. Error bars show 95% confidence interval.

FIGS. 16A and 16B show the effect of hydralazine on clearance of aggregated mutant huntingtin protein (EGFP-HD103Q). FIG. 16A is a confocal analysis showing the clearance of aggregated EGFP-HD103Q in stable inducible HeLa cells expressing EGFPHD103Q. Cells were treated with hydralazine or untreated (as control) for 48 hours. The fraction of EGFP-positive cells expressing aggregates under different conditions were plotted as odds ratios taking Ctrl value as 1. Error bars show 95% confidence interval. Rapamycin (10 μM) was used as positive control. FIG. 16B is a Western blots analysis of soluble EGFP-HD103Q species (mono and aggregate) in stable inducible HeLa cells expressing mutant EGFP-HD103Q. Cells treated with hydralazine same as above. Rapamycin (10 μM) was used as positive control. Right panel is densitometry analysis of EGFP-HD103Q levels normalized to actin. Control condition is set to 100%. Error bars show SD.

FIGS. 17A to 17C show the protective properties of hydralazine against Aβ (1-42) induced cytotoxicity. FIG. 17A plots the viability of SH-SY-5Y cells treated with Aβ (1-42) (20 μM) and hydralazine (5, 10 and 20 μM) for 24 hours. Cell viability was measured using cell titer glow luminescence assay. The data represent as percent cell viability normalized to untreated cells. The values are mean percent of n=2 performed in triplicates. Asterisks and signs denote values that are significantly different from those obtained from control cells; *P<0.05, **P<0.01. FIG. 17B shows the effect of hydralazine on GSK3β phosphorylation in Aβ (1-42) treated SH-SY5Y cells. SH-SY5Y cells were treated with Aβ (1-42) (500 nM), hydralazine (10 μM), LiCl (10 mM), Aβ plus hydralazine (500 nM and 10 μM), and Aβ plus LiCl (500 nM and 10 mM) for 24 hours followed by western blot analysis with anti-GSK33 and p-GSK33 antibodies. Quantification results show that the level of inactive phosphorylated GSK3β was significantly increased in hydralazine treated cells compared to the control and Aβ (1-42) treated cells. Densitometry values were normalized against actin. FIG. 17C shows the effect of hydralazine on p-Tau (S396) in Aβ (1-42) treated SH-SY5Y cells. The levels of p-Tau and total Tau were measured by Western blot analysis. Quantification results show a significant decrease in the level of phosphorylated Tau in hydralazine treated cells compared with the controls. Densitometry values were normalized to actin using Image J software. All of the western blot quantification values (shown in the right panel) are mean±SD from two technical replicates (n=2).

FIGS. 18A to 18D show the effect of hydralazine on p-GSK3 and p-Tau level in Aβ (1-42) treated primary neurons. FIGS. 18A and 18B show an immunofluorescence analysis of primary neurons isolated from mouse cortex treated with 1 μM hydralazine in the presence and absence of 500 nM A3-42. An increase in p-GSK33 level (A, green) and a decrease in the level of p-Tau (B, green) compare to controls were observed. Cells were transfected with tdTomato (red) plasmid to visualize structural and morphological changes of neurons. Nuclei were stained with DAPI (blue) scale bar=10 μm. FIG. 18C shows a Western blot analysis of the levels of p-GSK33 and total GSK3β. Inactive p-GSK33 was increased in cells treated with hydralazine compared to the control cells. FIG. 18D Western blot analysis of the p-Tau (S396) with PHF1 antibody. The p-Tau level showed significant decrease in cells treated with hydralazine compared to control cells. Densitometry values were normalized using actin as an internal control. Western blot (18C-18D) data plots shown in the right panels are mean±SD from two technical replicates.

FIGS. 19A to 19C show the protective properties of hydralazine against Aβ (1-42) induced toxicity in HEKTau^{P301S} cells. FIG. 19A shows the viability of HEKTau^{P301S} cells measured with and without 20 μM Aβ (1-42) and/or 5 and 10 μM hydralazine treatments for 24 hours. Cell viability was measured using cell titer glow luminescence assay. The data represents as percent cell viability normalized to DMSO treated control cells. The values are mean percent of two biological experiments performed in six replicates. Asterisks and signs denote values that are significantly different from those obtained from control cells; (**P<0.001, ****P<0.0001). FIG. 19B shows the effect of hydralazine on p-Tau and p-GSK3β levels in Aβ (1-42) treated mutant FL-Tau overexpressing HEK293 cells. HEK293FLTau^{P301S} cells were treated with 500 nM Aβ (1-42) in the presence or absence of 10 μM hydralazine for 24 hours. After treatment Western blot analysis was performed to check the levels of p-GSK3β and total GSK3β in addition to p-Tau and total Tau level. Hydralazine treatment increased inactive p-GSK33 and decreased the p-Tau level significantly compared to controls. Densitometry values were normalized using actin as internal control. All western blot analysis (FIG. 19B, 19C) values are mean±SD from three biological independent experiments (shown in the right panel). FIG. 19C shows the effect of hydralazine on GSK3 phosphorylation in Aβ (1-42) treated HEK293 cells.

FIGS. 20A to 20C show the effect of hydralazine on neuronal plasticity. Primary neuronal cells from cortex were treated with 0.5, 1.0 and 2 μM of hydralazine for 24 hours followed by confocal microscopy. Treatment with hydralazine markedly increased the number of spines (FIG. 20A) and other structural features (FIG. 20B) compared to control. FIG. 20C shows an experimental representation of spines on the dendrites. For each group (control, 0.5 μM Hyd, 1 μM Hyd, 2 μM Hyd and 2 μM Dimebon) 10 images from three replicates were analyzed. Total numbers of spines, stubby, thin, and mushroom were counted by NeuronStudio software for each image. The mean value and standard deviation of numbers for each group were calculated. P value was calculated with non-parametric T-test. All values are mean±SD (*p<0.05, **p<0.01).

FIG. 21A to 21C show the effect of hydralazine on dendrite spine density and morphology in neurons treated with neurotoxic amyloid β-42. Primary neuronal cells from cortex were visualized with td-Tomato (FIG. 21A). Cells were treated with 500 nM Aβ-42 oligomers or vehicle (Ctrl). Spine density and morphology were measured per 10 micron by confocal imaging. The overall spine density was reduced with Aβ-42 treatment compared to the control. But treatment for 24 hours with 1 μM hydralazine in presence of 500 nM of Aβ-42 increased the number of spines compared to Aβ-42 treated neurons (FIG. 21A, 21B). Mushroom and thins are indicated with an arrow. Treatment with Aβ-42 significantly reduced mushroom (MS) and thin (T) densities but increased the stubby (S) density compared to Ctrl. Treatment with hydralazine, on the other hand, decreased the stubby density while increasing mushroom and thin densities (FIG. 21C) compared to Aβ-42 treated cells bringing spine densities closer to Ctrl neurons. Dimebon, a known neuroprotective compound, was used as positive control in all experiments. Data were collected from three batches of cultures for all treatments and Ctrl. For spine quantification n=8-10 (for each treatment per one experiment) neurons were analyzed by Neuronstudio. Scale bar corresponds to 5 μm. Experiment was repeated three times. Values are shown as mean±SEM. (*p<0.05, **p<0.01, ***p<0.001, ****p<0.0001).

FIGS. 22A to 221 show the structures for the active agents that prevent neurodegeneration or that treat neurodegeneration of neural cells of the present invention. FIG. 22A is hydralazine, FIG. 22B is 1-Hydrazinyl-4-(prop-2-yn-1-yloxy)phthalazine, FIG. 22C is 1,2-Dimethylhydralazine, FIG. 22D is 4-Hydrazinylphthalazin-1-ol, FIG. 22E is 1-Chloro-4-hydrazinylphthalazine, FIG. 22F is 4-Chlorophthalazin-1-ol, FIG. 22G is Phthalazin-1(2H)-one, FIG. 22H is 6-Hydrazinyl-2-methyl-[1,2,4]triazolo[5,1-a]phthalazine, FIG. 22I is Isonicotinohydrazide.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Every organism inevitably experiences “aging”, a biological fate that progressively deteriorates the physiological function of the organism. This includes the onset of sarcopenia (muscle mass loss), cardiovascular disease, dementia, and neurodegeneration, all of which significantly affect the quality of life for older people. In the last few decades, there has been an increase in the number of people affected by neurodegenerative disorders, mainly due to a dramatic increase in life expectancy, which has led to a demographic change towards an older population. Degenerative diseases of the nervous system, including Alzheimer's disease (AD), Parkinson's disease (PD), and Huntington's disease (HD), impose substantial medical and public health burdens on populations throughout the world, particularly in the United States with its aging Baby Boomer population. As the prevalence of these diseases rises dramatically with age, the number of their cases is expected to increase for the foreseeable future as life expectancy increases around the world. According to the National Institute of Health, more than five million Americans are currently living with AD and at least 500,000 Americans live with PD, although some estimates are much higher. In fact, the current year's report claims that nearly 500,000 new cases of AD will be diagnosed this year alone. Despite these serious threats to the community, few effective treatments are available to combat these deadly diseases. Therefore, it is of tremendous importance to provide new therapeutic targets and strategies to avoid the onset and progression of deadly diseases like Alzheimer's, Parkinson's and Huntington's.

In last two decades, most laboratories and biotech companies are actively searching for compounds to prevent neuronal loss and restore memory and cognitive function in diseases like AD, PD, and HD.

Despite an observation of positive responses to compounds in various disease non-vertebrate and vertebrate models, none of these efforts have resulted in a treatment or cure due to failures of candidate drugs in clinical trials.

Maintaining a good quality of proteins is one of the fundamental strategies used by every living organism (from yeast to human) for keeping normal cellular function. To restore cellular homeostasis, several machineries (e.g., repair, detoxification, refolding, and degradation) have evolved to maintain good quality of proteins. If any of these networks are compromised (often seen in aging and neurodegeneration), proteins will undergo misfolding and form higher-order structures (oligomers to aggregates), which become pro-oxidant and induce cell death. In fact, multiple studies using non-vertebrate and mouse models have shown a direct cause and effect relationship between neurodegeneration and aggregation when toxic, aggregation-prone polypeptides such as stretch of poly glutamate residues (poly Q), mutant superoxide dismutase 1 (SOD1), and A1 (1-42) peptide, are overexpressed. On the other hand, dietary restriction (40% calorie restriction) or other genetic manipulations that are known to extend the lifespan of both non-vertebrate and mouse models (i.e., down regulation of insulin growth factor 1 (IGF-1) or expression/overexpression of heat shock factor-1 (Hsf-1)) prevent the formation of toxic aggregates in neurodegenerative models and restore physiological function (cognitive function for mice, locomotor activity for C. elegans). There are some reports in literature that showed that dietary restriction had either no effect on lifespan extension or had deleterious effects on longevity. However, dietary restriction is still considered to be the only experimental manipulation that typically shows beneficial effects on longevity and healthy aging.

Substantial evidences in the literature now strongly suggest that aging is an inevitable prelude and a compounding factor for the onset of neurodegenerative diseases like Alzheimer's and Parkinson's, because as animals age most of the defense machineries weaken. Most of the drugs or immunological strategies that showed promising effects in mouse and other non-vertebrate neurodegenerative models are mainly designed to prevent the formation of or disrupt the existing oligomers and aggregates of the target proteins (e.g., Aβ (1-42), α-synuclein, mutant SOD1). However, none of these strategies have proven effective in improving the cellular environment by preventing accumulation of toxic aggregates. The present invention manipulates the cellular environment in such a way that the defense systems that are compromised in diseases are restored and that the accumulation of toxic elements is removed by the activation of degradation machinery.

The present invention is based on the discovery of the protective and rejuvenating property of the drug hydralazine (Hyd), and related active derivatives (e.g., 1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, is Phthalazin-1(2H)-one, 6-Hydrazinyl-2-methyl-[1,2,4]triazolo[5,1-a]phthalazine, Isonicotinohydrazide), in neuronal cells. The protective properties of Hyd, and its active derivatives, are usually attributed to its ability to chelate toxic protein carbonyls. There are many different known mechanisms for production of protein carbonyls in cells (i.e., formation of various lipid peroxidation aldehyde adducts as a result of ROS modifications of lipids that can in turn covalently modify proteins or more direct formation of protein carbonyls as a result of lysine, arginine and threonine side chains oxidation by ROS). Protein carbonyls are highly reactive and can react with any primary amine in cell environment (i.e., lysine side chains or proteins amino terminus) to form cross-linked proteins that can eventually lead to proteins aggregates which are a hallmark of neurodegenerative diseases. As an explanation, but not a limitation of the present invention, the protective properties of hydralazine and its related active derivatives may be the result of its carbonyl chelating capabilities. As shown herein, this carbonyl chelating activity was demonstrated by challenging neuronal-like PC12 cells with hydrogen peroxide (H₂O₂), a ROS generating molecule that can be produced endogenously as a result of mitochondrial metabolism, in the presence and absence of hydralazine and a similar chelator hydrazine (Hy) as a positive control.

### Example 1: Hydralazine Protects Cells from Oxidative Stress Independent of Carbonyl Group Chelation

Briefly, the present inventors found that Hydralazine protects cells from oxidative stress independent of carbonyl group chelation. It was also found that Hydralazine activates the Nrf2/SKN-1 pathway. Hydralazine protected Alzheimer's disease model cells and C. elegans from chemical stressors linked to neurodegenerative diseases. Finally, it was found that Hydralazine extends C. elegans life and healthspan in a SKN-1 (Nrf2 ortholog) dependent manner.

One of the main mechanisms underlying compromised physiological function in aging and age-related diseases is chronic elevation of reactive oxygen species (ROS) (Bokov et al., 2004; Dias et al., 2013). Because oxidative damage is a direct threat to cell survival several important defense machineries (i.e. ROS scavengers, repair and refold machineries and degradation apparatus) have evolved to maintain cellular homeostasis. When these defense machineries are severely compromised, as observed in aging and age-related diseases (i.e. Alzheimer's (AD), Parkinson's (PD), Huntington's disease (HD), etc.) cell function is misregulated and oxidative stress-induced cell death is accelerated (Douglas and Dillin, 2010; Stadtman, 1992).

One of the master regulatory elements modulating a diverse set of defense machineries against oxidative stress is nuclear factor erythroid 2-related factor 2 or Nrf2 (Itoh et al., 1999; McMahon et al., 2001). Nrf2 regulates more than 200 genes encoding cytoprotective phase II detoxification and antioxidant enzymes, including HO-1, NQO-1, glutamate-cysteine ligase subunits (GCLc and GCLm) and glutathione-S-transferase (GST) which collectively synthesize glutathione (GSH) to assist in maintaining GSH over the oxidized form GSSG (Lu, 2009; Sheehan et al., 2001). GSH reduces ROS such as superoxide and hydroxyl radicals non-enzymatically and acts as an electron donor for the reduction of peroxides in a glutathione peroxidase-catalyzed reaction (Brannan et al., 1980).

Under normal conditions, Nrf2 is sequestered in the cytosol by a Keap1 (Keltch-like ECH associated protein 1) homodimer. The half-life of Nrf2 is short (˜15 min) as it is ubiquitinated and degraded rapidly by the proteasome machinery (McMahon et al., 2006; Tong et al., 2006). When cells are stressed, however, a conformational change is induced in Keap1, mediated by three reactive cysteine residues (C151, C273 and C288), resulting in the release of Nrf2 (Bryan et al., 2013). Once released, Nrf2 scrapes the Cul3 mediated degradation pathway which increases its half-life to 60 min. Free Nrf2 is then phosphorylated at Ser-40 by protein kinase Cwhich triggers the translocation of p-Nrf2 into the nucleus (Huang et al., 2002). Nrf2 then rapidly enters the nucleus, forms a complex with Maf2, and binds to antioxidant respond element (ARE) sequences in the upstream promoter regions of many antioxidative genes (Kwak et al., 2003; Thimmulappa et al., 2002).

To develop a molecular probe for identification of carbonylated proteins in brain the present inventors searched for a molecule that 1) reacts with protein carbonyls efficiently, 2) crosses the blood-brain barrier, and 3) has a suitable structure for attachment of a purification handle. The inventors selected hydralazine as an ideal candidate. It was unexpectedly discovered that this drug, FDA-approved for the treatment of hypertension, has anti-aging properties. It is demonstrated herein that hydralazine activates the Nrf2 signaling pathway. Using in vitro and in vivo model systems (SH-SY5Y cells, primary neuronal cells, and C. elegans) that inventors show that hydralazine treatment activates cyto-protective elements, quantified by measuring cell viability, cytotoxicity and animal survival, by triggering the translocation of Nrf2 from cytoplasm to nucleus followed by ARE activation. Additionally, the inventors show that in both in vitro and in vivo models, hydralazine protects against chemical stressors such as rotenone. Finally, the inventors show that hydralazine extends lifespan in C. elegans dependent on SKN-1, the worm Nrf2 ortholog. Activation of Nrf2 by hydralazine provides a protective mechanism to shield neuronal cells that are otherwise vulnerable in a compromised environment that elicits aging and diseases such as AD and PD.

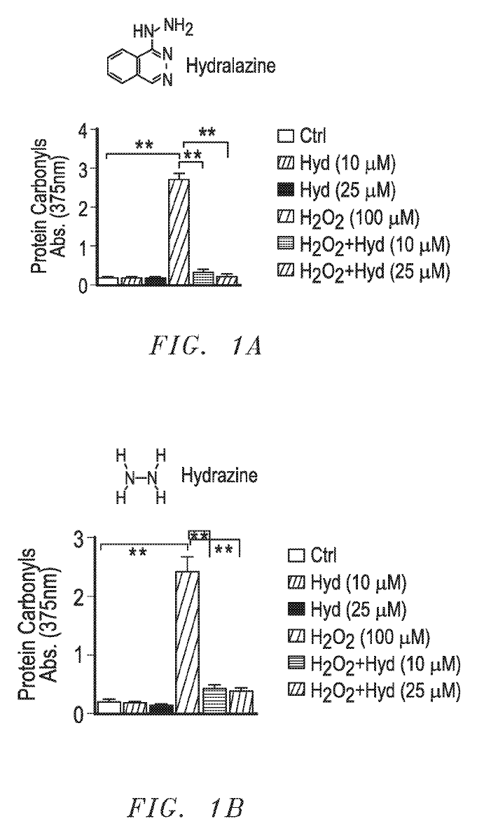

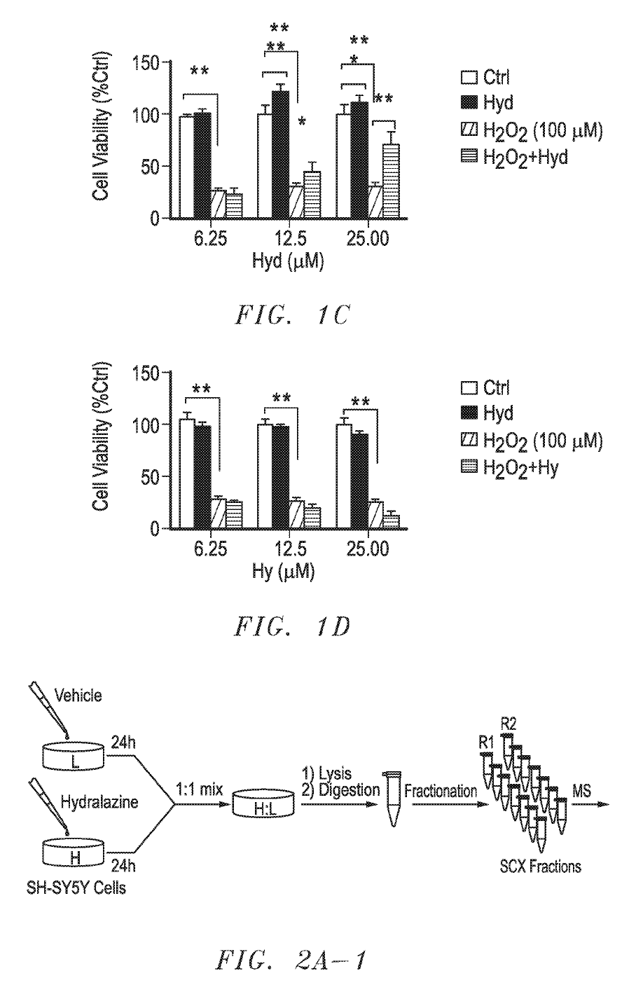

Hydralazine protects cells from H₂O₂ induced cell death independent of carbonyl group chelation. In addition to its utility in the treatment of hypertension, hydralazine was shown to inhibit acrolein-mediated injuries in ex vivo spinal cord via acrolein aldehyde functional group chelation (Hamann et al., 2008). Considering the importance of aldehyde toxicity and the potential benefits of identifying carbonylated proteins, the inventors first tested the reactivity of hydralazine (Hyd) as an aldehyde chelator. To generate intracellular aldehydes, the inventors treated human neuroblastoma cell line (SH-SY5Y) with 100 μM hydrogen peroxide (H₂O₂) for 24 h. Carbonyl groups were quantified using 2,4-DNPH (dinitrophenylhydrazine) assay. Hydrazine, a compound with the same functional group as hydralazine, was used as a positive control. Control and stressed cells were both treated with 10 and 25 μM of hydralazine or hydrazine (FIGS. 1A-1B). Both hydrazine and hydralazine reduced protein carbonyls significantly. Surprisingly, when the inventors assayed cell viability using an 3-[4,5-dimethylthiazol-2-yl]-2,5-diphenyltetrazolium bromide; thiazolyl blue (MTT) assay under the same experimental conditions, hydrazine failed to protect cells from H₂O₂ induced cell death whereas hydralazine provided protection in a dose-dependent manner (FIG. 1C-D). This result suggested that the protection of the stressed cells by hydralazine was not the result of carbonyl chelation.

Hydralazine activates the Nrf2/SKN-1 pathway. The inventors then determined the mechanism(s) underlying hydralazine's mode of action by performing a global comparative proteomics screen using stable isotope labeling with amino acids in cell culture (SILAC) (FIG. 2A) (Ong et al., 2002). SH-SY5Y cells grown in light and heavy media were treated with 0 and 10 μM hydralazine respectively, and, after 24 h, cells were collected and lysed. Equal amounts of lysate protein were combined, digested, and analyzed by shotgun mass spectrometry which resulted in quantification of ˜5,400 proteins. The SILAC results were subjected to Ingenuity™ Pathway Analysis (IPA) which implicated activation of the Nrf2 pathway (Z score=0.156, p value=1.97E-10) among others (FIG. 2C). The inventors pursued Nrf2 over other pathways because Nrf2 controls over 200 genes that are directly or indirectly involved in maintenance of cellular homeostasis resulting in improved cell survival and extended lifespan.

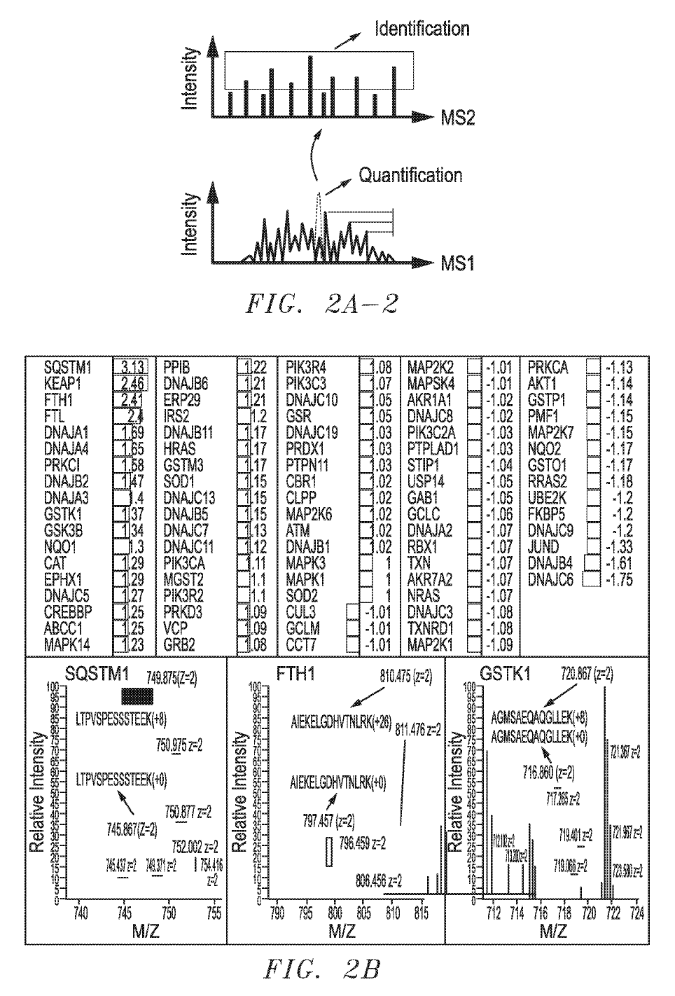

The inventors quantified Nrf2 protein in treated cells as a first step towards validating the prediction of the Nrf2 pathway. Even though Nrf2 level by itself may only have a mild impact on Nrf2 pathway activation (there are several other regulatory mechanisms). SH-SY5Y cells were treated with hydralazine (0, 10 μM) for 24 h and Nrf2 was detected by Western blotting, which revealed that hydralazine caused Nrf2 upregulation (about 20% increase in Nrf2 level compared to untreated cells) (data not shown). The inventors next investigated the effect of hydralazine on the interaction between Nrf2 and Keap1, next step in Nrf2 pathway activation, by reciprocal Nrf2-Keap1 and Keap1-Nrf2 co-immunoprecipitations. Hydralazine significantly reduced the Nrf2-bound Keap1 in the anti-Nrf2 antibody pull down (30-40% reduction treated compared to control). Similarly, the Keap1-bound Nrf2 was significantly reduced in the presence of hydralazine (30% reduction) in the anti-Keap1 antibody to pull down (FIG. 3A).

Having established that Nrf2-Keap1 binding was reduced, the inventors examined Nrf2 nuclear localization, the next step in Nrf2 pathway activation, by performing subcellular fractionation and Nrf2 partition quantification in SH-SY5Y cells. In cells treated with hydralazine (10, 20 μM), the inventors determined that nuclear localization of Nrf2 was significantly increased, while the cytosolic Nrf2 fraction remained unchanged (FIG. 3B). Nrf2 phosphorylation on serine 40 is a regulatory modification required for Nrf2 translocation to nucleus and downstream protein activation. As shown in FIG. 3C, the phosphorylation of Nrf2 on serine 40 in the nuclear compartment was increased (25%) by hydralazine, while no change was observed in phosphorylation of cytosolic Nrf2. The inventors used lamin and actin respectively as markers for purified nuclear and cytosolic fractions and sulforaphane, a known Nrf2 activator, caused similar changes in phosphorylation and nuclear localization of Nrf2 (FIGS. 3B, 3C).

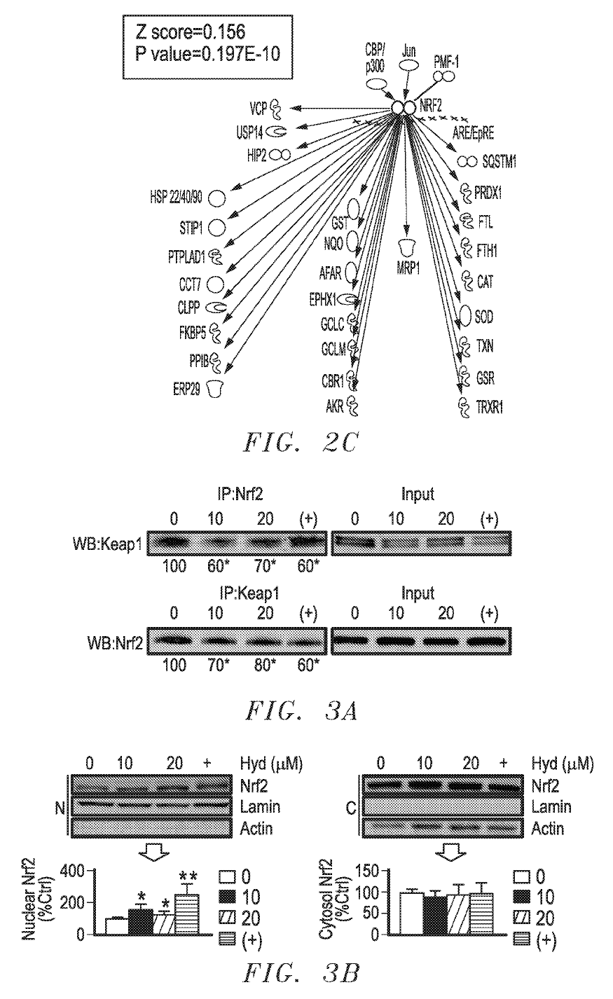

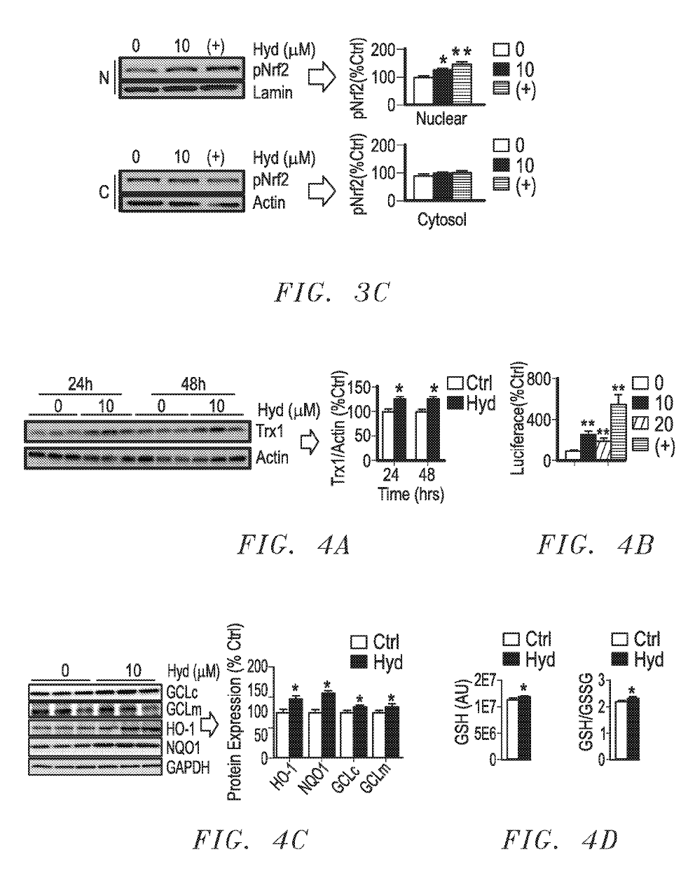

Next, the inventors measured the relative amounts of thioredoxin 1 (Trx1) in untreated and hydralazine-treated cells. Trx1 reduces the cysteine residues critical for binding of Nrf2 to AREs (Hansen et al., 2004). The data presented in FIG. 4A shows that the Nrf2-mediated activation of ARE. Hydralazine increased the amount of Trx1 by 25% after 24 h and 48 h. To determine the functional relevance of the increases in expression, translocation and phosphorylation of Nrf2, the inventors next measured Nrf2 transcriptional activity using a luciferase-based ARE-controlled gene expression system. SH-SY5Y cells expressing the ARE-luciferase reporter were treated with hydralazine for 24 h prior to harvest. Both concentrations of hydralazine increased luciferase activity significantly compared to untreated cells (2.0±0.2 folds) (FIG. 4B).

To further confirm Nrf2 activation, the inventors next measured the expression of Nrf2 downstream targets, GCLc, GCLm, HO-1, and NQO-1 in SH-SY5Y cells treated with hydralazine for 24 h. By Western blotting, these four targets showed a significant increase in expression (FIG. 4C). HO-1 and NQO-1 showed upregulation of 30% and 40% respectively. To determine the result of increasing expression of these enzymes, the inventors measured GSH and the GSH/GSSG ratio. GSH is regulated by the rate-limiting enzyme GCL consisted of GCLc and GCLm subunits. The inventors quantified the total GSH and GSSG using a luciferase-based assay in SH-SY5Y cells treated with hydralazine for 24 h. As shown in FIG. 4D, 10 μM hydralazine increased GSH and pushed the GSH/GSSG ratio slightly (5%) higher but significantly towards the reduced form of glutathione (GSH). These results further confirmed the upregulation of GCLc and GCLm with hydralazine treatment. These data collectively support the hypothesis generated by the quantitative proteomics screen that identified hydralazine as an activator of the Nrf2 pathway. To establish a direct link between Nrf2 activation and hydralazine-mediated protection against oxidative stress, the inventors knocked down Nrf2 to determine the efficacy of hydralazine in rescuing cells with reduced Nrf2 from H2O2-induced cytotoxicity. Nrf2 was depleted with a targeted siRNA (FIG. S2). The inventors then used extracellular lactate dehydrogenase cytotoxicity assay to measure cell survival. Nrf2 knockdown SH-SY5Y cells experienced more cytotoxicity from H₂O₂ than control cells (2.2-fold versus 1.8-fold) and no protection form hydralazine (FIG. 4E).

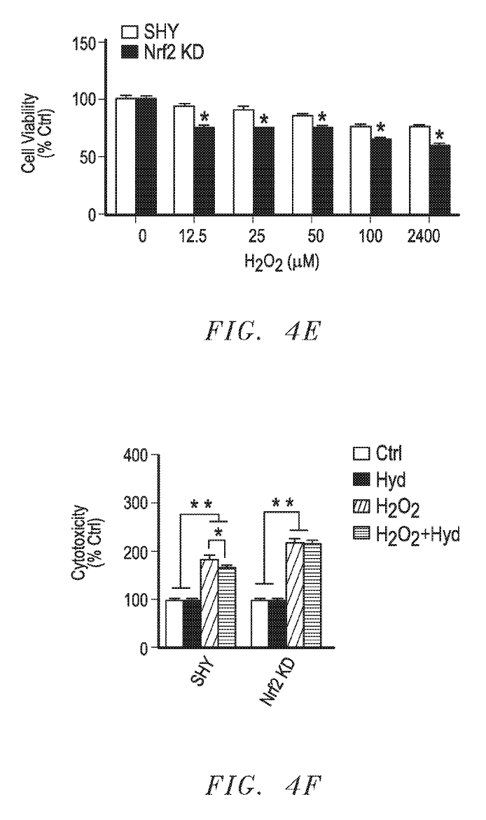

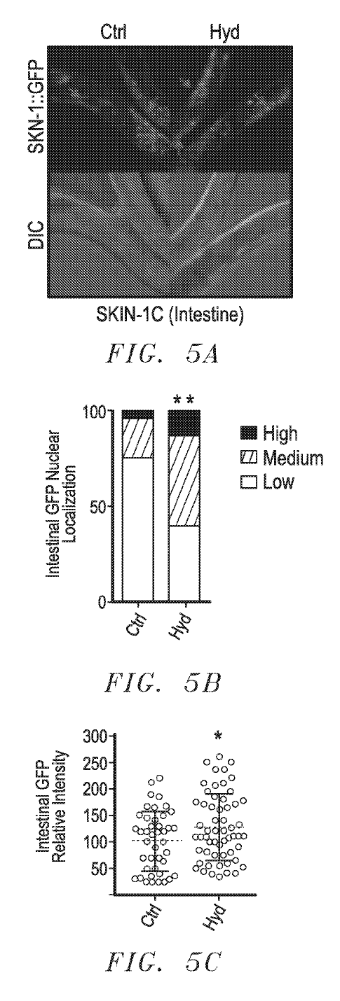

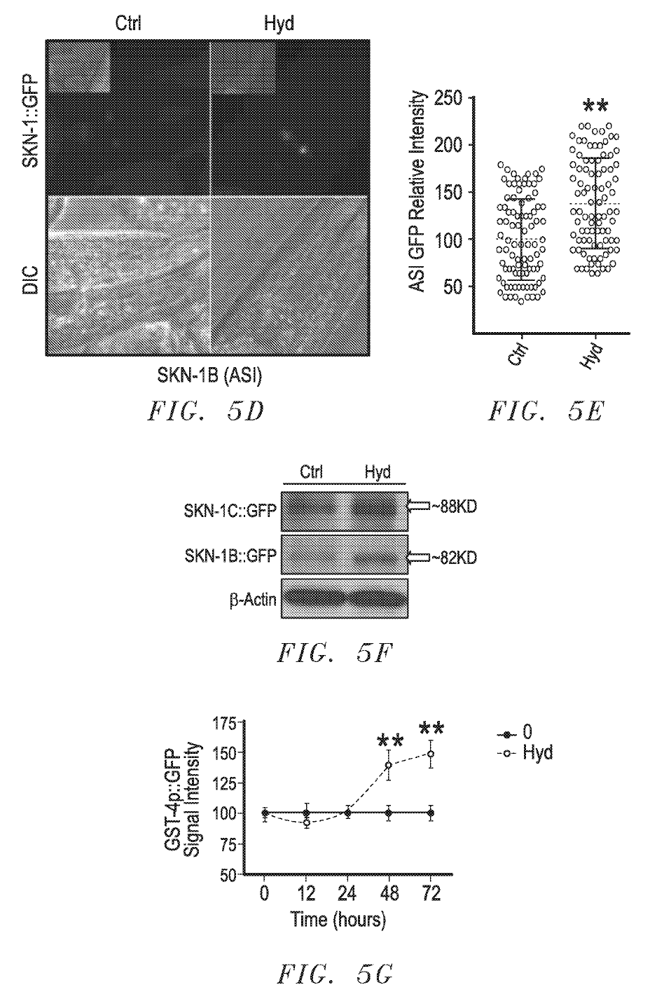

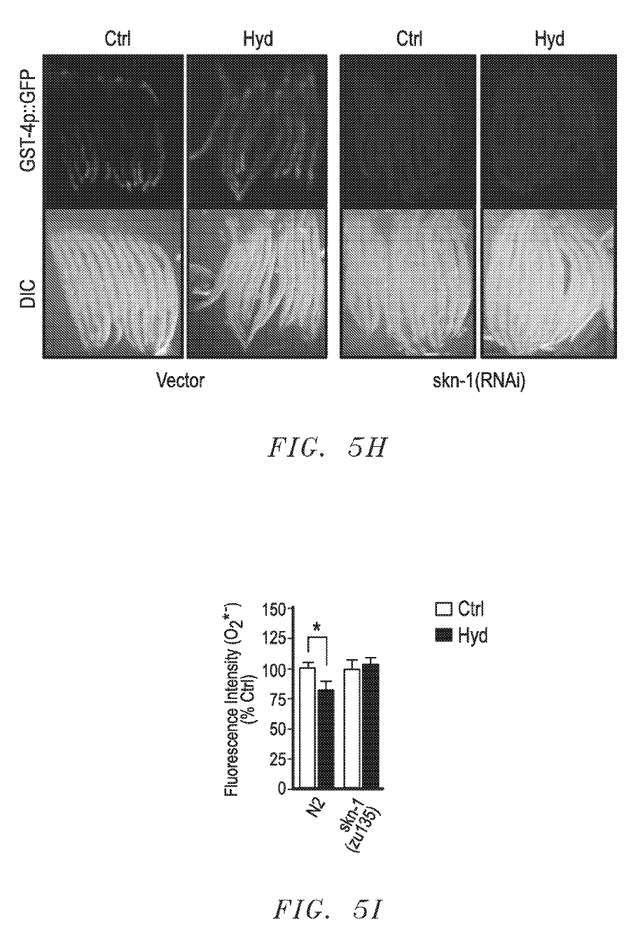

The C. elegans Nrf2 ortholog, SKN-1, shows remarkable functional conservation relative to its mammalian counterpart making C. elegans an ideal model for in vivo studies (An and Blackwell, 2003; Blackwell et al., 2015). SKN-1 is primarily expressed in the intestine where it regulates oxidative stress. It is also expressed in ASI chemosensory neurons (putative hypothalamus) where it mediates the longevity benefits of dietary restriction (DR) (Bishop and Guarente, 2007; Blackwell et al., 2015). Hydralazine treatment significantly increased the signal intensity and localization of SKN-1::GFP in the intestinal nuclei and the signal intensity in ASI neurons of C. elegans (FIGS. 5A-E). The expression of both SKN-1 isoforms (B and C) measured by Western blot showed increases with hydralazine treatment confirming the data acquired by imaging (FIG. 5F). The inventors measured the expression of glutathione S-transferase-4 (GST-4), a downstream target of SKN-1, in a transgenic strain (CL2166) expressing GFP driven by the GST-4 promoter (Kahn et al., 2008). Hydralazine treatment caused a significant increase in the GFP signal 48 h after treatment (data not shown). But hydralazine treatment did not increase GFP signal in worms fed skn-1(RNAi) or lacking a functional intestinal skn-1 isoform c in a mutant strain (skn-1(zu67)) (FIG. 5H and data not shown). The inventors measured superoxide concentration in N2 and skn-1(zu135) mutant treated with hydralazine. Hydralazine treatment decreased superoxide concentration in N2 worms but not in mutant worms (FIG. 5I). To further support hydralazine-induced SKN-1 activation, the inventors conducted a global comparative proteomics analysis. Synchronized populations of N2 normal C. elegans strain were treated for 3 days with 100 μM of hydralazine or vehicle. Treated and untreated populations, four biological replicates each, were then lysed, digested and analyzed by shotgun mass spectrometry for label-free quantification. A total of 3,113 proteins were detected across all biological replicates of which 269 proteins were downregulated and 143 were upregulated. An IPA analysis was performed using proteins with human orthologs. The SKN-1/Nrf2-stress response pathway was found activated with a significant score (Z score=3.317, p-value=0.0002) (FIGS. 5J and S4). Red dots in the volcano plots represent components of the SKN-1 pathway. The SKN-1 pathway human orthologs, represented with red dots in the volcano plot and their log 2 fold change (FC), are also shown in FIG. 5J.

Hydralazine-mediated Nrf2/SKN-1 activation provides neuroprotection. To evaluate the potential therapeutic value of hydralazine the inventors determined two important disease-related questions, 1) will hydralazine-mediated protection from H₂O₂ toxicity translate to protection against cytotoxicity present in neurodegenerative conditions? 2) is the extent of hydralazine-induced activation of Nrf2 sufficient to protect cells with compromised defense systems? To answer these questions the inventors used WT primary neuronal cells and HEK293 cells overexpressing tau residues 244 to 372, with mutations of P301L and V337M exposed to recombinant tau fibrils that indefinitely propagate tau aggregates (aggregate-positive cells). Exact same HEK293 cells grown in the absence of tau aggregate exposure only propagating tau RD monomers were used as control (aggregate-negative cells) (Sanders et al., 2014). The inventors used rotenone as an exogenous source of stress to further challenge both primary and HEK293 cells.

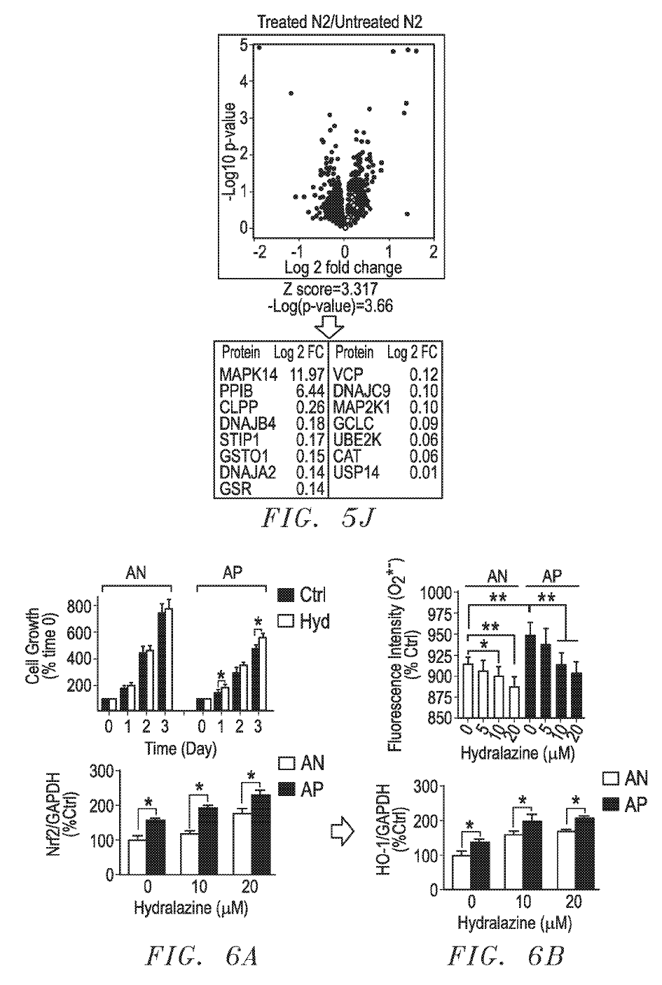

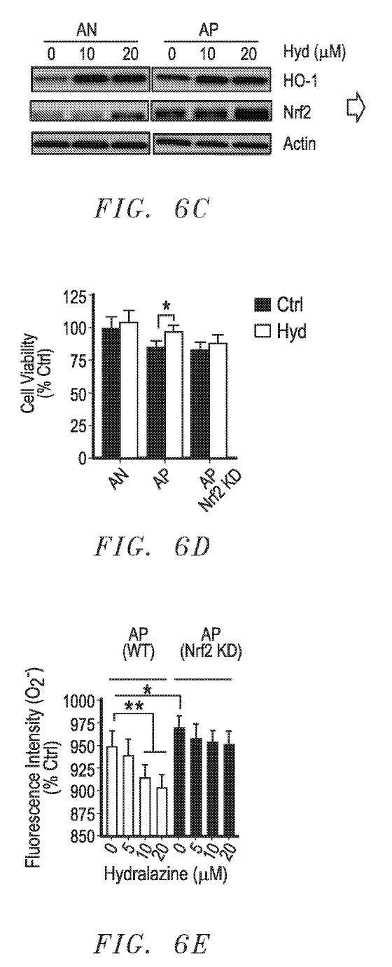

The inventors first measured the cell proliferation to evaluate the stress caused by expression of tau fibrils in aggregate-positive and negative cells (FIG. 6A). Cell growth was significantly impeded in aggregate-positive cells and improved with hydralazine treatment. The inventors also measured the concentration of superoxide ions (O2-), a detrimental byproduct of oxidative phosphorylation, as an ROS representative. The concentration of superoxide decreased in a dose-dependent manner with hydralazine treatment in aggregate-positive and -negative cells (FIG. 6B). The inventors next measured expression of Nrf2 and HO-1 in the same cells (FIG. 6C). As expected the Nrf2 expression was higher (1.5 fold). HO-1 expression, on the other hand, only exhibited a modest increase (˜25%), suggesting only a small functional response to increased Nrf2 expression (FIG. 6C). The inventors knocked down Nrf2 to determine if hydralazine-mediated protection was Nrf2 dependent (data not shown). Aggregate-positive and -negative KD cells were treated with 200 μM H₂O₂ for 24 h. Cytotoxicity was measured as release of LDH. Knockdown of Nrf2 rendered cells more sensitive to H₂O₂ and aggregate-positive KD cells exhibited higher sensitivity compared to aggregate-negative KD cells (data not shown). Hydralazine treatment did not improve cell viability in KD cell line in a significant manner (FIG. 6D). No significant change in the concentration of O2− ions was observed in aggregate-positive KD cells with hydralazine treatment (FIG. 6E). The inventors next sought to determine if hydralazine provided additional protection in aggregate-positive cells exposed to exogenous stress from rotenone, a pesticide and complex I inhibitor known to be causative for PD-like pathology in animal models (Sherer et al., 2003). Aggregate-positive and -negative cells were treated with 20 μM hydralazine in the presence or absence of 1 μM of rotenone for 24 h and subjected to Western blot analysis and cell viability assays. Nrf2 expression was higher in aggregate-positive cells treated with hydralazine and rotenone compared to cells treated with rotenone only (FIG. 6F). Cell viability assays showed that hydralazine protects both aggregate-positive and negative cells from impairment caused by rotenone (FIG. 6G). The inventors also treated primary cortical neuronal cells with rotenone as a disease model to study hydralazine-mediated protection. Hydralazine rescues primary neuronal cells from rotenone toxicity in a dose-dependent manner (FIG. 6H).

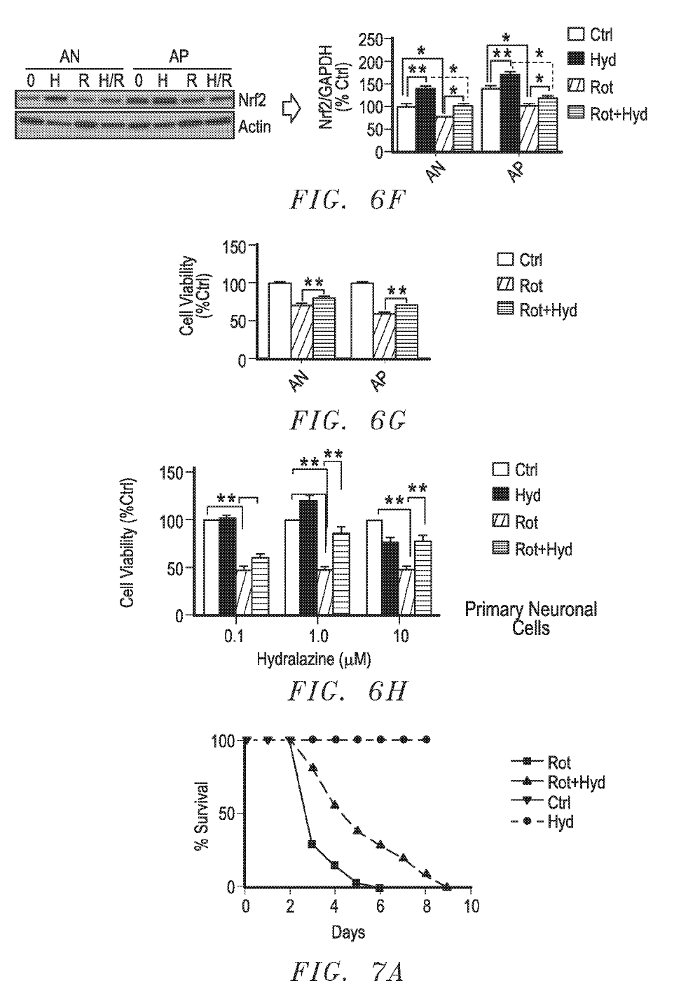

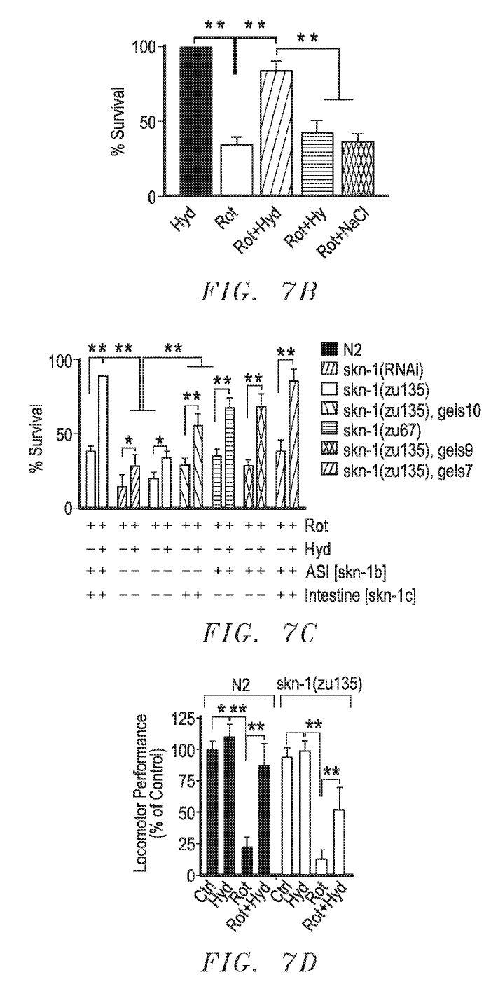

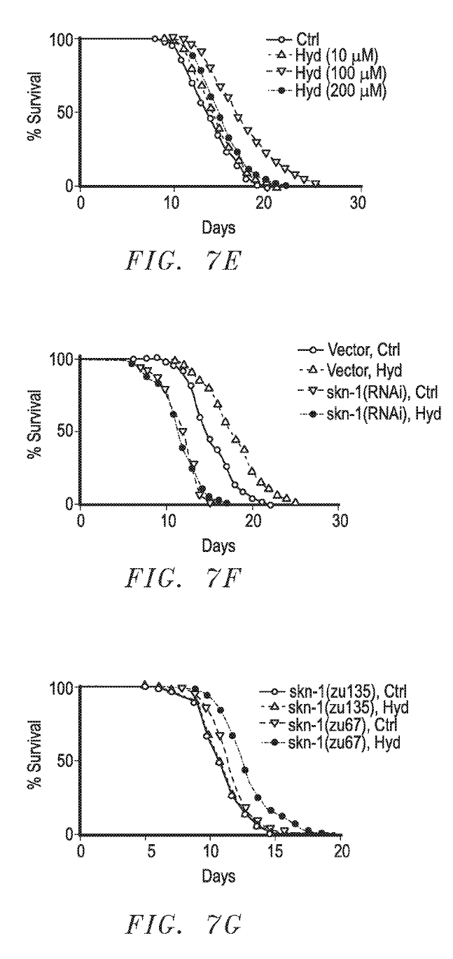

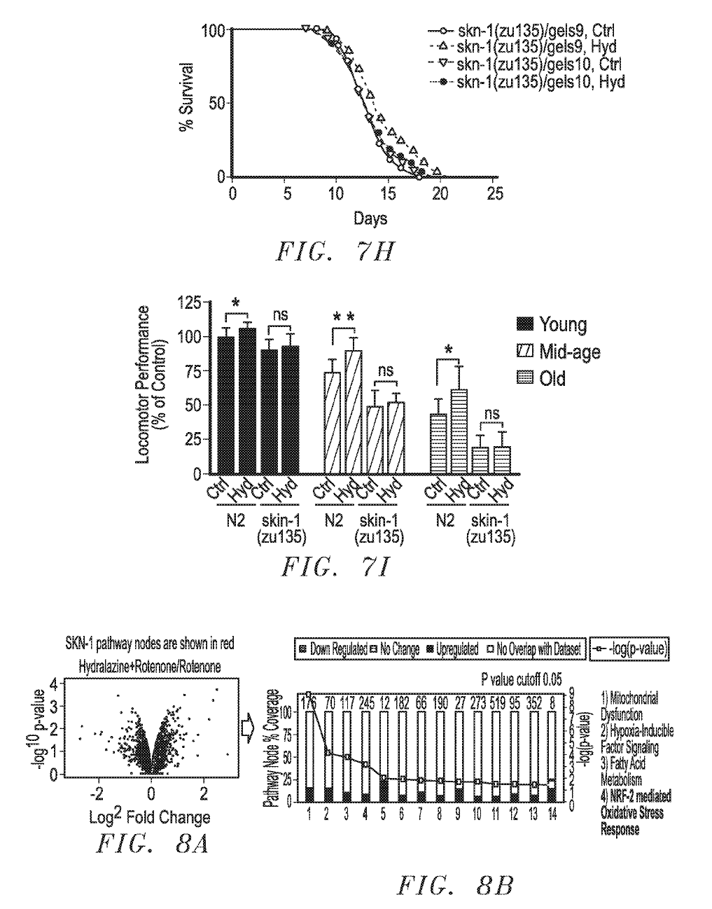

The inventors assessed the in vivo capacity of hydralazine to reduce oxidative stress by evaluating its efficacy in protecting C. elegans from rotenone-induced neurotoxicity. SKN-1 plays a key role in C. elegans antioxidant machinery; thus, the inventors anticipated that hydralazine would be neuroprotective (Blackwell et al., 2015). The inventors again used rotenone to elicit PD-like changes. After preconditioning adult worms for 48 h with hydralazine (100 μM), worms were exposed to rotenone (50 μM) and their viability was measured. Most of the rotenone-treated animals died in 24 h; however, hydralazine treated animals were significantly protected (p<0.0001) against rotenone-induced death (FIG. 7A). No protection against rotenone toxicity was observed in worms treated with 100 μM NaCl or hydrazine (FIG. 7B). The inventors also performed a global comparative proteomics analysis using label-free mass spectrometry to identify pathways activated with hydralazine in worms under rotenone stress. Based on IPA results, SKN-1/Nrf2 pathway was fourth from the top amongst 14 activated stress response pathways in worms treated with hydralazine and rotenone compared to worms treated only with rotenone (data not shown). In worms treated with rotenone compared to control worms SKN-1/Nrf2 was not among the activated pathways (data not shown). However when the inventors compared hydralazine-treated rotenone-stressed animals to the control group, SKN-1/Nrf2 was found seventh amongst 13 activated stress response pathways indicating suppression of SKN-1/Nrf2 pathway in rotenone treated worms (data not shown).

To understand the dependence on different SKN-1 isoforms in hydralazine-mediated neuroprotection, the inventors used a loss-of-function skn-1 mutant and transgenic animals that mosaically express skn-1 isoform b in chemosensory neurons and skn-1 isoform c in the intestine. In skn-1(zu135) mutant, which has loss of function inactivation of SKN-1, hydralazine protection sharply decreased compared to N2 C. elegans (FIG. 7C). Similar results were obtained when skn-1 was knocked down confirming the skn-1(zu135) mutant results. However expression of skn-1 isoforms b or c partially restored hydralazine-mediated protection. In skn-1(zu67) with an active isoform b and inactive isoform c, better protection was observed compared to the skn-1(zu135), highlighting the importance of isoform b upregulation in ASI neurons in hydralazine-mediated protection against rotenone. But activation of both isoforms in transgene Is007 results in maximum protection against rotenone-induced cytotoxicity by hydralazine, suggesting a cooperative role for both isoforms (FIG. 7C).

The inventors also evaluated locomotion in worms to investigate the health of the rescued animals. The results showed superior locomotor performance of the hydralazine pre-treated animals exposed to rotenone compared to the control group (p<0.007) (FIG. 7D). In skn-1(zu135) mutants, the better locomotor performance of hydralazine-treated worms exposed to rotenone was reduced (FIG. 7D).

Hydralazine extends C. elegans life and healthspan in a SKN-1 dependent manner. To determine if the improved stress resistance associated with exposure to hydralazine extends the life and health span, the inventors directly measured the effect of hydralazine on these characteristics in C. elegans (Bishop and Guarente, 2007; Blackwell et al., 2015; Castillo-Quan et al., 2016). A synchronized population of N2 normal worms was grown for their entire lifespan on medium containing hydralazine from 10 to 200 μM. Lifespan was extended by as much as 25% in animals on 100 μM hydralazine (p<0.0001) (FIG. 7E). The inventors assessed the role of SKN-1 activation in the pro-longevity effects of hydralazine. The pro-longevity effect of hydralazine was completely lost in skn-1(zu135) mutant and skn-1 knockdown worms but not in worms fed with control vector (FIGS. 7F-7G). However in mutant skn-1(zu67) worms with a functional SKN-1 isoform B, a less robust extension of lifespan was observed in worms treated with hydralazine. Using transgenic animals with mosaic expression of isoform b in the ASI neurons (geIs9) or isoform c in the intestine (geIs10) the inventors found that skn-1 isoform b is critical for the prolongevity effects of hydralazine (p<0.0005) (FIG. 7H). Although the presence of skn-1 isoform c does not have a significant impact on lifespan extension, its availability along with isoform b is necessary to achieve maximum lifespan extension.

Hydralazine also resulted in a significant improvement in the locomotor performance of young (5 days), middle age (10 days), and old (15 days) N2 animals (FIG. 7I). To study the role of SKN-1 in hydralazine-mediated extension of healthspan further, the inventors treated mutant skn-1(zu135) animals the same way as N2 wild type and measured their locomotion at three time points, young (4 days), middle age (8 days) and old (12 days) (FIG. 7H). Locomotor performance did not improve in skn-1(zu135) animals with hydralazine treatment at any time demonstrating the role of SKN-1 in delaying age-dependent deterioration of locomotion in C. elegans.

Table 1 shows the number of trials and population sizes for lifespan studies in FIGS. 7A to 71.

| | | Median | Variation | | |
|---|---|---|---|---|---|
| Related to | | Lifespan | Compared to | P-values against | |
| figure | Strains and Conditions | SE (days) | Control (%) | Control | n (trials) |
| 7E | N2OIJM | 13.80.15 | | | 473 (5) |
| | N210 IJM Hyd | 14.50.35 | +4.82 | ns | 168 (2) |
| | N2100IJMHyd | 17.20.15 | +24.4 | P value < 0.0001 | 483 (5) |
| | N2200 IlM Hyd | 15.60.50 | +13.3 | P value < 0.0019 | 176 (2) |
| 7F | N2-Scramble Rr, IAi- | 13.50.35 | | | 166 (2) |
| | O IJM | | | | |
| | N2-Scramble RNAi- | 16.50.35 | +22.2 | P value < 0.0001 | 164 (2) |
| | 100 IJM | | | | |
| | N2-skn-1 RNAi-O IJM | 11.00.70 | | | 183 (2) |
| | N2-skn-1 RNAi- | 10.51.00 | −5.4 | ns | 186 (2) |
| | 100 μM | | | | |
| 7G | EU31Crll | 10.00.33 | | | 270 (3) |
| | EU31-100 IlM Hyd | 10.30.19 | +3.3 | ns | 282 (3) |
| 7G | EU1Clrl | 11.3:t0.69 | | | 278 (3) |
| | EU1100 IJM Hyd | 12.6:t0.83 | +11.7 | P value < 0.0055 | 293 (3) |
| 7H | LG357Clrl | 120.20 | | | 292 (3) |
| | LG357-100 FM Hyd | 12.30.33 | +2.7 | ns | 303 (3) |
| 7H | LG348Clrl | 12.30.50 | | | 253 (3) |
| | LG348100 IJM Hyd | 13.60.70 | +10.8 | P value < 0.0055 | 245 (3) |

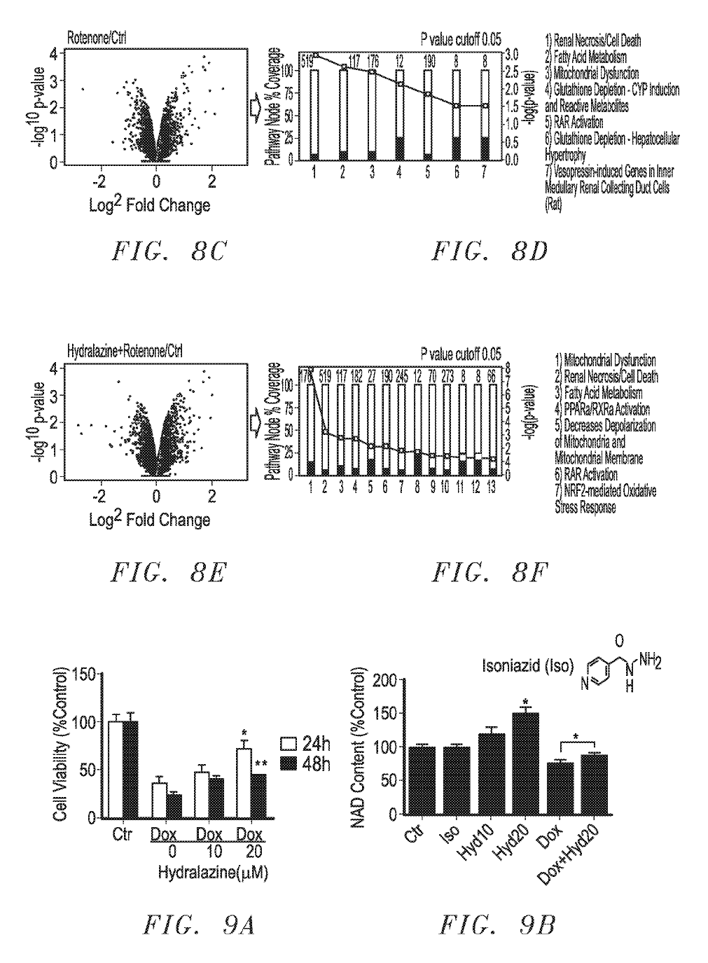

FIGS. 8A to 8F show that Hydralazine activates SKN-1/Nrf2 pathway in worms treated with rotenone. FIG. 8A is a Volcano plot showing the ratio (hydralazine+rotenone/rotenone) distribution of proteins quantified by label-free mass spectrometry. SKN-1/Nrf2 pathway nodes are shown in red. FIG. 8A shows the proteins ratios obtained by label free mass spectrometry were uploaded for IPA analysis. SKN-1/Nrf2 was number four in the top five activated stress response pathways (p-value cutoff of 0.05). FIG. 8C is a Volcano plot showing the ratio (rotenone/Ctrl) distribution of proteins quantified by label-free mass spectrometry. SKN-1/Nrf2 pathway nodes are shown in red. FIG. 8D shows the results of IPA analysis for worms treated with rotenone compared to control. SKN-1/Nrf2 was not among activated pathways (p-value cutoff of 0.05). FIG. 8E is a Volcano plot showing the hydralazine+rotenone/Ctrl ratio distribution of proteins quantified by label-free mass spectrometry. SKN-1/Nrf2 pathway nodes are shown in red (p-value cutoff of 0.05). FIG. 8F shows that the SKN-1/Nrf2 pathway was among activated pathways when worms treated with rotenone and hydralazine were compared to control worms (p-value cutoff of 0.05).

Nrf2 inactivation has been linked to aging and age-related disorders. In Hutchinson-Gilford progeria syndrome (HGPS), a fatal premature aging disorder, the Nrf2 pathway is repressed and increased oxidative stress that results from Nrf2 pathway inactivation was proven to be sufficient to induce HGPS aging defects (Kubben et al., 2016). Furthermore, reactivation of the Nrf2 pathway in cells from HGPS patient reverses aging defects and restores in vivo viability of mesenchymal stem cells in an animal model (Kubben et al., 2016).

In neurodegenerative diseases, neurons need an optimal GSH supply to defend themselves against free radicals released from activated microglia and astroglia. Maintaining GSH requires activation of the Nrf2 pathway (Steele and Robinson, 2012). In the hippocampus, one of the brain areas where neurodegeneration starts (Ramsey et al., 2007), astrocytes from AD patients have less Nrf2 than normal. Decreased glutathione has been reported in the substantia nigra of individuals with PD. Even though Nrf2 is localized to the nucleus in the neurons that survive in the substantia nigra, it is not known if the Nrf2 transcription machinery is functional (Ramsey et al., 2007). In addition, mutations in the nrf2 gene have been linked to both AD and PD progression (von Otter et al., 2010a; von Otter et al., 2010b). Nrf2 is also reduced in motor neurons of the spinal cord and cortex of ALS patients (Sarlette et al., 2008). These studies demonstrate that the Nrf2 system is impaired in individuals suffering from neurodegenerative diseases and significant health benefits result from restoration or activation of the Nrf2 pathway.

These studies demonstrate that therapies aimed at Nrf2 activation provide protection that benefits neurons experiencing age-related degenerative conditions. In this study, the inventors demonstrate that hydralazine, an FDA approved drug, to be an activator of Nrf2 pathway with potential therapeutic value for the treatment of neurodegenerative diseases and general improvement in age-related pathologies. As such, Hydralazine is like other FDA approved drugs such as metformin and rapamycin, which promote healthy aging.

The inventors also showed that the protection SH-SY5Y cells experience with hydralazine treatment is not related to the drug's aldehyde chelating properties, even though the chelating properties of hydralazine are additionally beneficial when combined with its Nrf2 activating propensity. By measuring protein carbonyls and viability of cells treated with hydralazine the inventors showed that not only hydralazine does not cause oxidative stress, it increases cell viability under normal conditions. Comparing protein carbonyl concentration in cells under H₂O₂ stress treated with hydralazine and hydrazine the inventors concluded that the presence of benzodiazine rings in hydralazine does not accelerate its metabolism and it may even stabilize the compound. To unravel hydralazine's mechanism of action the inventors used an unbiased proteomic screen to identify cellular pathways modulated by hydralazine treatment (FIGS. 2A to 2C). As predicted, multiple pathways were found activated by hydralazine including eIF2 signaling, protein ubiquitination, mTOR signaling, insulin receptor signaling, AMPK signaling, and the Nrf2 pathway. The inventors pursued Nrf2 pathway due to its direct role in oxidative stress response and significant p value. Crucial steps in the Nrf2 activation process, including dissociation of Nrf2 from Keap1, Nrf2 phosphorylation and translocation to the nucleus, ARE binding and activation, and up-regulation of stress response elements were monitored in SHY cells to confirm the activation of the Nrf2 pathway by hydralazine. Hydralazine-mediated activation of Nrf2 might also explain its antihypertensive effect. The inventors demonstrate that the antihypertensive effect of hydralazine is mediated by Nrf2 pathway activation.

One of the critical determinants of Nrf2 activation is Trx1 (Hansen et al., 2004), which facilitates the binding of Nrf2 to Adenylate-uridylate-rich elements (ARE). Trx1 is a redox sensor, which plays a role in maintaining neuronal health and its overexpression results in extended lifespan in mice (Perez et al., 2011). The inventors examined Trx1 in SH-SY5Y cells after hydralazine treatment and also quantified ARE binding capacity. As anticipated based on the results taught hereinabove, Trx1 was increased by 25%. ARE binding was also elevated by 2-fold by hydralazine. The inventors next checked enzymes downstream in the Nrf2 pathway, HO-1, NQO-1 and two subunits of GCL. These enzymes are important responders to stress; in particular, GSH the product of the GCL-catalyzed reaction contributes to protein thiol homeostasis. Further proof was provided by showing that more GSH shifts to its oxidized form GSSG with hydralazine treatment. To explore the dependence of hydralazine-mediated neuroprotection on Nrf2, the inventors knocked down Nrf2 in SH-SY5Y cells and showed unequivocally that hydralazine-mediated neuroprotection is Nrf2 dependent.

As a means of explanation, and in no way a limitation of the present invention, there are three possible mechanisms for hydralazine-mediated activation of Nrf2 pathway, (i) shifting cellular environment towards a mild pro-oxidant state and altering the conformation of Keap1 to release Nrf-2 from Keap1-Nrf2 complex, (ii) direct binding and interruption of Keap1-Nrf2 interaction, and finally (iii) activating Nrf2 via PI3K/Akt pathway. Based on the intensity of the oxidative stress measurement probe DCFH-DA (Dichloro-dihydro-fluorescein diacetate) (data not shown), hydralazine does not create a mild pro-oxidant environment. Results from Keap1-Nrf2 inhibitor screening assay ruled out the direct binding hypothesis (data not shown). However, proteomics data and pAkt/Akt ratio measurement by Western blot suggests that Akt mediated phosphorylation of Nrf2 might be one of the underlying mechanisms for Nrf2 pathway activation (data not shown).

The inventors chose C. elegans as a well-known model to address the in vivo relevance of the discovery of the stress-protective actions of hydralazine. During postembryonic stages, the Nrf2 ortholog, SKN-1, regulates phase II detoxification genes through constitutive and stress-inducible mechanisms in ASI chemosensory neurons and the intestine, respectively. SKN-1 is present in ASI nuclei under normal conditions, and accumulates in intestinal nuclei in response to oxidative stress. The inventors showed that hydralazine upregulates SKN-1, increases its nuclear localization, activates the downstream target GST-4, and reduces the concentration of superoxide in treated worms. Global comparative proteomics was also used to confirm the activation of SKN-1 pathway.

Hydralazine was also effective on a tauopathy cell model and chemically induced PD cell models, elevating Nrf2 and HO-1 expressions, reducing superoxide concentration, and consequently protecting cells from stress induced by tau aggregate by a mechanism primarily dependent on Nrf2. Because many neurodegenerative diseases are multifactorial disorders (Clinton et al., 2010), the inventors challenged tauopathy model cells with rotenone to mimic conditions closer to what cells may experience under neurodegenerative conditions and showed hydralazine protects cells from multiple sources of stress (i.e., tau aggregates and rotenone).

In this study, the inventors chose HEK293 cells forming tau fibrils as tauopathy disease model cells. One advantage of this model was that it had a perfect control, the exact same cell line that did not form tau fibrils despite expressing the same double mutant tau. In all the studies performed on the aggregate-positive cells the inventors observed a significant response. In many neurodegenerative conditions Nrf2 expression is suppressed and as a result hydralazine treatment is expected to have much larger impact. Another possibility is the level of stress that these cells are experiencing in presence of tau fibrils. If this stress is much higher than the stress neurons experience in the brains of tauopathy patients, the observed hydralazine effect may be underestimated. To demonstrate this effect, the inventors used primary neuronal cells treated with rotenone as a model for PD (Radad et al., 2008). Hydralazine treatment protected primary neuronal cells from rotenone toxicity increasing their viability much more significantly back to levels comparable to untreated cells.

Using a C. elegans model, hydralazine extended lifespan and healthspan following challenge with rotenone, again by mechanisms largely dependent on the worm Nrf2 ortholog. In response to a high concentration of rotenone pathways related to cell death, rather than Nrf2, dominated the IPA analysis of the proteomic data. Again, hydralazine mitigated the rotenone response, activating the SKN-1/Nrf2 pathway. Treatment with hydrazine or NaCl did not protect worms from rotenone toxicity ruling out general stress response as the underlying mechanism. skn-1 mutants are sensitive to oxidative stress and have shortened lifespans (25%-30%) (An and Blackwell, 2003; Edwards et al., 2014; Park et al., 2009; Uno and Nishida, 2016). At one week of age, small cavities and yolk droplets appear in the heads of skn-1 mutant but not wild-type animals. The anterior intestine and posterior pharynx also degenerate more frequently before death in skn-1 mutant animals compared to wild type. These changes are typical of aging C. elegans, suggesting premature aging in skn-1 mutants (Garigan et al., 2002). On the other hand, increased longevity in diet-restricted C. elegans has been attributed to skn-1 activation in the ASI neurons, which signal peripheral tissues to increase metabolic activity (Bishop and Guarente, 2007). Considering the importance of SKN-1 in C. elegans aging, the inventors chose this model to test the effects of hydralazine treatment on worm's lifespan. The data herein show that hydralazine extends both medium and maximum lifespan of worms treated with hydralazine by at least 25%. SKN-1 was also essential for this action of the drug.

Lifespan and healthspan are two different equally important subjects that are not necessarily mutually inclusive. It has been shown that ASI neurons mediate SKN-1 induced longevity by an endocrine mechanism and affect non-neuronal body tissues non-autonomously (Bishop and Guarente, 2007). Hydralazine improved the locomotor performance of N2 nematoda at all ages (young, mid-age and old) but not in skn-1(zu135) highlighting the importance of SKN-1 activation in extension of healthpsan in C. elegans.

Again, not intending to be bound by theory, and in not way a limitation of the present invention, while it seems that hydralazine-mediated extension of lifespan and healthspan in C. elegans is primarily SKN-1 dependent, a consideration of these data leaves open the possibility that hydralazine-mediated protection against stress involves other mechanisms. In skn-1(zu135) worms, hydralazine-mediated protection in the presence of rotenone was decreased as a result of SKN-1 inactivation; yet some protection was still observed. Similarly, the locomotor performance of skn-1(zu135) worms treated with rotenone and hydralazine was improved but to a lesser extent compared to N2 animals. In summary, the inventors have shown using a variety of model systems and assessments that hydralazine mitigates the impact of cellular stresses on neuronal models. Its actions may offer significant health benefits for patients with neurodegenerative conditions (Park et al., 2014).

Materials and Methods. Chemicals. All the chemicals were purchased from Sigma (St. Louis, Mo.) unless otherwise stated in the text.

Cell culture. Neuroblastoma SH-SY5Y cells were purchased from ATCC (ATCC® CRL-2266™) and maintained in DMEM medium supplemented with 10% fetal bovine serum. The cells were cultured in a humidified chamber at 37° C. with 5% CO2. Cells were plated the day before the treatment so that the density of cell culture could reach approximately 70% confluence. Hydralazine was diluted in culture medium from a stock solution. The final concentration of hydralazine and the duration of the treatment were indicated in the text and the Figure legends. Oxidative stress was induced in cells with different concentrations of stressors (e.g., hydrogen peroxide or rotenone (mitochondrial complex I inhibitor)) to test the efficacy of hydralazine. Hydrogen peroxide treatment was done in 5% serum containing medium.

At the end of the treatment, cells were collected and washed once in ice-cold PBS buffer, followed by lysis with RIPA buffer (1% Triton X-100, 1% sodium deoxycholate, 0.1% SDS, 0.15M NaCl, 0.01M sodium phosphate, pH7.2) supplemented with cocktails of proteases and phosphatases inhibitors (Thermo Fisher, Waltham, Mass.) on ice for one hour with occasional stirring. The cell lysates were then centrifuged at 10,000 g for 15 minutes at 4° C. Supernatants were collected and protein concentration was measured using the BCA method.

Protein carbonyl Assay. Protein carbonyls were measured using a commercially available kit (MAK094; Sigma-Aldrich, St. Louis, Mo.). Cells were first lysed in lysis buffer provided by the kit.

Following centrifugation, 100 μL of supernatant was added to 10 μL of 10% streptozocin solution, was incubated at room temperature for 10 min and re-centrifuged to remove interfering nucleic acids. Carbonyl content of supernatant was determined by adding 2, 4-dinatrophenylhydrazine followed by detection of dinitrophenyl hydrazine adduct at 375 nm in a 96 well plate reader. The protein content of each sample was determined using a bicinchoninic acid (BCA) assay, Thermo Fischer, Waltham, Mass.).

Cell culture in SILAC media and Lysate Preparation. SHY-SY5Y cells were grown in Dulbecco's modified Eagle's medium containing either unlabeled L-Proline, L-arginine (Argo) and L-lysine (Lyso) or L-Proline, heavy isotope-labeled L-arginine-¹³C6¹⁴N₄(Arg¹⁰) and L-lysine-¹³C6-¹⁵N2 (Lys⁸) (Cambridge Isotope Laboratories, Inc.) supplemented with 10% dialyzed fetal bovine serum (Thermo Fischer, Waltham, Mass.). Light labeled cells were left untreated to serve as control and heavy labeled cells were treated with 10 μM of hydralazine for 24 h. After treatment, cells were harvested by trypisinization, washed three times with cold PBS and lysed in a buffer containing 6M Urea, 2M Thio-urea, 1% SDS and 100 mM Tris/HCl, pH 8.0 with protease (Thermo Fischer, Waltham, Mass.) and phosphatase inhibitors. After incubation for 15 min at RT and sonication, the samples were clarified by centrifugation for 15 min at 20,000×g. Protein content was determined using the 660 nM protein assay kit (Thermo Fisher, Waltham, Mass.) according to the manufacturer's instructions.

Protein Digestion and Peptide Fractionation. Equal amounts of protein from control and hydralazine treated cells were mixed in 1:1 ratio and digested in solution. The mix digest (300 μg) was then fractionated into six fractions via strong cation exchange (SCX). SCX cartridges were pre-equilibrated with a buffer composed of 0.5% acetic acid and 2% ACN (wash buffer). The digest was then loaded onto the column and washed with wash buffer and subsequently eluted with a buffer containing ammonium acetate (30 mM, 50 mM, 70 mM, 80 mM, 120 mM and 500 mM), 0.5% acetic acid, and 2% ACN. Eluted peptide fractions were desalted using reverse phase cartridges.

Mass Spectrometry for SILAC. All the fractions were analyzed using a Q-Exactive HF mass spectrometer (Thermo Electron, Burlingame, Calif.) coupled to an Ultimate 3000 RSLCnano HPLC systems (Thermo Electron, Sunnyvale Calif.). Peptides were loaded onto a 75 μm×50 cm, 2 μm Easy-Spray column (Thermo Electron, Sunnyvale Calif.) and separated using a 120 min linear gradient from 1-28% acetonitrile at 250 nl/min. The Easy-Spray column was heated at 55° C. using the integrated heater. Shotgun analyses was performed using a data-dependent top 20 method, with the full-MS scans acquired at 60K resolution (at m/z 350) and MS/MS scans acquired at 15K resolution (at m/z 200). The under-fill ratio was set at 0.1%, with a 3 m/z isolation window and fixed first mass of 100 m/z for the MS/MS acquisitions. Charge exclusion was applied to exclude unassigned and charge 1 species, and dynamic exclusion was used with duration of 15 seconds.

Western Blot Analysis. Protein expression was determined by Western blot analysis. Equal amount of protein from each sample was run in Tris-glycine SDS PAGE gel, followed by transferring to PVDF membrane. After blocking the membrane with 5% milk for 1 hour at room temperature, the membrane was incubated further for 2 hours with antibodies specific for target proteins: Nrf2, pNrf2(S40) from Novus (Littleton, Colo.); Keap1, HO-1, NQO1, GCLc, GCLm and lamin B1 from Cell Signaling (Danvers, Mass.); GAPDH from Santa Cruz (Dallas, Tex.); β-Actin from Thermo Fisher (Waltham, Mass.). The membrane was subsequently incubated with species-specific HRP-conjugated secondary antibody followed by incubation with chemiluminescence substrate and imaging. The band intensity of each of the target proteins was quantified using ImageQuant software (GE Healthcare, Sweden). Western blot analysis on worm lystaes was done similarly. Young adult control and hydralazine treated (72 h) animals were collected, washed 3 times in M9, flash froze in liquid nitrogen and lysed in RIPA buffer with protease inhibitor by sonication. Equal amounts of protein were run on the gel and SKN-1 protein level was detected using anti-GFP antibody.

Cell Viability and Cytotoxicity Assay. Cell growth was analyzed using the MTT cell viability assay. Briefly, at the end of incubation/treatment, MTT reagent was diluted in culture medium and aliquoted into each well. After incubation for 2 hours, the medium was aspirated and DMSO was aliquoted into each well to disrupt the cells and dissolve the intracellular MTT dyes. Absorbance was read at 570 nm wavelength in a 96-well plate reader. Cell toxicity was detected using a LDH cell toxicity assay kit (Promega, Fitchburg Wis.). This is a coupled enzymatic assay that detects a visible color signal initially described by Nachlas et. al. The reagent contained excess concentrations of lactate and NAD+as substrates to drive the LDH reaction and produce NADH. In the presence of diaphorase, NADH drives the enzyme-catalyzed conversion of iodonitro-tetrazolium violet to a red formazan product. Briefly, DMEM medium was withdrawn from each well of the plate after treatment and briefly centrifuged to get rid of floating cells or cell debris. Then, the medium supernatant was mixed with equal volume of substrate/enzyme mixture included in the kit (lactate, NAD+, diaphorase and iodonitro-tetrazolium violet), followed by incubation at room temperature for 15 minutes before the addition of acetic acid to cease the reaction. The absorbance was read at 490 nm in a 96-well plate reader. Primary cortical neuronal cells were cultured in 96 wells plates for three weeks then treated with 0.1, 1.0, and 10 μM of hydralazine or 1 μM of rotenone, or 0.1, 1, and 10 μM of hydralazine in presence of 1 μM of rotenone for 24 hours. Cell viability assay was performed using CellTiter-Glo assay.

Co-immunoprecipitation (Co-IP). Cells were lysed in ice-cold 50 mM Tris-HCl buffer with 0.5% Triton X-100 and protease inhibitors, followed by centrifugation at 12,000 g for 15 minutes at 4° C. Protein in the supernatant from each of the samples was incubated with antibody in 1:1 ratio (either specific for Nrf2 or Keap1), at 4° C. for overnight, on a rotator with constant stirring. Protein A/G magnetic beads (Thermo Fisher, Waltham, Mass.) were added into the antibody-antigen mixture followed by incubation for 1 hour at 4° C. on a rotator. The tube was applied to a magnetic stand to collect the beads, followed by washing in lysis buffer for three times. Finally, the bead-bound antibody-antigen mixture was eluted with equal volume of 1× electrophoresis sample buffer. The eluted protein was subjected to Western blot analysis as described earlier.

Cell Fractionation. Nuclear and cytoplasmic fractions were separated using the protocol and NE-PER Nuclear and Cytoplasmic Extraction Reagents provided by Promega (Fitchburg Wis.). Briefly, the cells were harvested and washed with ice-cold PBS buffer. The cells were resuspended in ice-cold CER-I buffer and incubated on ice for 10 minutes, followed by addition of CER-II buffer and incubation for one more minute on ice. The lysate was centrifuged at 16,000 g for 5 minutes. The supernatant was the cytoplasmic fraction. The pellet obtained was lysed in ice-cold NER buffer to obtain nuclear protein fractions.

Nrf2 Knockdown by siRNA Transduction. Nrf2 was knocked down using a human Nrf2 specific siRNA in a lentiviral vector (sc-37030-V, Santa Cruz, Calif.). A scrambled siRNA was used as negative control. Transduction was conducted using a lentiviral transduction kit from Santa Cruz. Briefly, 50% confluent cells were made for transduction. During transduction, cells were kept in polybrene-containing complete medium, followed by aliquoting siRNA lentiviral particles into the medium. Stable clones were selected using 0.8 μM of puromycin for 2 weeks after 2 days of transduction. Expression of Nrf2 was determined by Western blot analysis as described earlier in this method and by quantitative real-time reverse PCR (qRT-PCR). Briefly, total RNA was isolated from the cell using Trizol RNA isolation reagents (Thermo Fisher, Waltham, Mass.) followed by reverse transcription of RNA to cDNA using a cDNA synthesis kit (Ambion, Austin, Tex.). Quantitative PCR was set up and run in an ABI 9700 model system (Applied Biosystems, Foster City, Calif.), using hNrf2-specific primers and equal amount of template cDNA.

Nrf2 Transcriptional Activity Assay. The transcriptional activity of Nrf2 was determined using a luciferase-based transcription activation approach. A vector carrying a Nrf2 promoter controlled luciferase gene (firefly luciferase) and a vector carrying the control luciferase (Renilla luciferase) from an ARE reporter kit (BPS Bioscience, San Diego, Calif.) was transiently co-transfected into the cells using Lipofectamine reagents (Thermo Fisher, Waltham, Mass.). After transduction for 24 hours, the cells were treated with hydralazine for another 24 hours before subjected to the luciferase assay with the Dual-Glo Luciferase system (Promega, Fitchburg Wis.). Briefly, the cells were incubated with firefly luciferase substrate for 10 minutes prior to measurement luminescence in a 96-well luminescence plate reader. Subsequently, the Renilla luciferase was measured after the addition of Dual-Glo Stop & Glo reagent into the wells with a 10-minute incubation. The ratio of luminescence from firefly and Renilla was calculated to normalize and compare the Nrf2 transcriptional activity.

Measurement of GSH/GSSG. The intracellular GSH and GSSG content were determined using a GSH/GSSG-Glo Assay kit (Promega, Fitchburg Wis.) following the manufacturer's instructions. Briefly, after treatment, half of the cells were lysed using total lysis buffer and the other GSSG lysis buffer for 5 minutes. Luciferin generation reagents (luciferin-NT plus Glutathione-S-Transferase) were then added into each well, followed by incubation for 30 minutes. Finally, luciferin detection reagents including Ultra-Glo™ Luciferase were aliquoted into each well. After 10-minute incubation, luminescence was read in a 96-well plate reader. GSH level was calculated by subtracting GSSG level from the total, and GSH/GSSG ratio was calculated as ([Total]−[GSSG])/[GSSG].

Measurement of Reactive Oxygen Species. Superoxide concentration was measured by dihydroethidium (DHE). Black clear bottom 96 well plates were seeded with about 5000 HEK293 tau aggregate-negative (control) and aggregate-positive model cells per well and about 20000 SH-SY5Y cells (the NRF2 knockdown of both cell lines were also seeded). Cells were treated with hydralazine and the superoxide level was measured by incubating cells with DHE (1 μM) for 30 minutes. Fluorescence was measured by Spectramax Gemini XPS plate reader (Molecular Devices, Sunnyvale, Calif.) at 370 nm excitation and 420 nm emission wavelengths. Superoxide concentration in worms was measured after hydralazine treatment for 3 days followed by Incubation with DHE (6 μM final concentration) for 30 minutes. Equal number of animals were transferred to a black bottom 96 well plate and fluorescence was measured same as above.

Protein Profiling Using Label-free Quantitation. Worms were lysed in 8M urea, 5% 1 M Tris-HCl pH 8.0 and 1× protease inhibitor cocktail EDTA-free (Thermo Fisher, Waltham Mass.) buffer with the aid of sonication. Lysates were centrifuged at 14000 g for 15 min at 4° C. and proteins in the supernatant were precipitated using cold acetone. Disulfide bonds were reduced and alkylated (by DTT and IAA respectively) before diluting the solution to 1.8 M with 25 mM TRIS-HCL pH 8.0. Proteins were digested overnight at 37° C. with trypsin (Promega, Fitchburg Wis.) in presence of 1 mM CaCl₂. Peptides were acidified with TFA and purified using Oasis HLB plates (Waters, UK). A Dionex Ultimate 3000 UHPLC (Thermo) was coupled to an Orbitrap Fusion Lumos mass spectrometer for the separation and analysis of tryptic peptides. An Easy-Spray column with 75 μM inner diameter and 50 cm long packed with 2 μM C18 material was used for peptide separation. 0.1% formic acid and 2% (v/v) acetonitrile in LCMS grade water was used as buffer A and 10% (v/v) TFA plus 80% (v/v) acetonitrile in LCMS grade water was used as buffer B. 5 μl of sample were injected and separated using a gradient from 0% to 28% mobile phase B over 180 min (240 min total run time). Source voltage was set to 2.2 kV and capillary temperature to 275° C. in the positive ion mode. Ions within the m/z range of 400-1600 were scanned at the resolution of 120,000. Collision induced dissociation method was used to fragment top 10 MS spectra with 2-7 charge states.

MS data processing and Ingenuity™ pathway analysis. LC-MS/MS raw data files were processed using the latest available MaxQuant software (v.1.5.3.30). Proteins were identified by the Andromeda search engine within the MaxQuant program and the search was performed against UniProt/Swiss-Prot Caenorhabditis elegans database. The inventors used one multiplicity as standard label free search. Carbamidomethyl cysteine was set as a fixed modification and methionine Oxidation (M) and Acetyl (protein N-term) were used as variable modifications. The protein and peptide false discovery rates and peptide-to-spectrum match (PSM) false discovery rate (FDR) were set to 1%. Match between runs was performed by using a match time window of 0.7 (minimum) and alignment time window of 20 (minimum). The decoy proteins, known contaminants (after quality control using cluster analysis), proteins identified with a single modified peptide and low confidence proteins identified by only one peptide were filtered out. The p-values for all the statistical analysis were calculated using a two-tailed student t-test as used for normally distributed data (we pre-processed intensities by binary logarithm). The inventors identified proteins with their fold change values and conducted Ingenuity Pathway Analysis (IPA) and the pathway analysis was performed by the Ingenuity Knowledge Base (genes only) as the reference set with direct and indirect relationships included.

C. elegans strains and maintenance. Animals were grown and maintained using standard C. elegans conditions at 20° C. on NGM plates and were fed E. coli strain HB101. N2 worms were used as wild-type and the following mutants and transgenic strains were used from Caenorhabditis Genetics Center (CGC, University of Minnesota): EU1 skn-1(zu67) (IV)/nT1[unc-?(n754); let-?], EU31 skn-1(zu135))/nT1[unc-?(n754); let-?], CL2166 dvIs19 [gst-4p::GFP::NLS], CL691 dvIs19 [gst-4p::GFP::NLS], skn-1(zu67)/nT [unc-?(n754) let-?], LG333 skn-1(zu135)/nT1[qIs51]; ldIs7[skn-1b/c::GFP], LG348 skn-1(zu135)/nT1[qIs51]; geIs9 [gpa-4p::skn-1b::GFP+rol-6(su1006)], LG357 skn-1(zu135)/nT1[qIs51]; geIs10 [ges-1p(long)::skn-1c::GFP+rol-6(su1006)], and LD1 ldIs7 [skn-1b/c::GFP+rol-6(su1006)].

Rotenone stress test. Hydralazine, hydrazine, and NaCl were dissolved in water, and rotenone was dissolved in DMSO. Synchronized L1 larvae were placed on NGM plates preloaded with hydralazine, hydrazine, or NaCl. After 3 days young adult worms were transferred to fresh NGM plates either preloaded with rotenone alone or rotenone plus any of the abovementioned compounds. Every worm was subjected to prodding test with a worm pick every day. A worm was scored as dead when not responding to three repeated proddings. Survival curve was plotted using Prism 7.

Lifespan analysis. All lifespan assays were performed at 20° C. according to standard protocols described elsewhere. Synchronized L1 animals were placed on either hydralazine or control NGM plates. Worms were transferred to fresh plates every day after reaching adulthood, and every two days after reaching 10 days of age. Prodding test as described above was used to count the number of dead worms. Survival curve was plotted using Prism 7 and the significance of the curves calculated by Log-rank (Mantel-Cox) test.

RNA Interference. Synchronized L1 larvae were placed on NGM plates containing 1 mM IPTG and fed HT115 bacterial strain containing scramble or skn-1 RNAi plasmids. All the experiments were done at 20° C.

Imaging. To measure the GFP intensity, synchronized populations of worms were anesthetized and arranged on agarose pad. The intestinal SKN-1::GFP was assayed by confocal microscopy (X40) (Nikon A1R, Nikon Instruments Inc., Melville, N.Y., USA) (FIG. 5A). The quantification of intestinal SKN-1::GFP was performed with ImageJ (FIG. 5B). The ASI SKN-1::GFP was assayed by a Zeiss Axiolmager M2 microscope equipped with a Hamamatsu Flash 4.0 Scientific c-mos camera and Zen2 software (X40) (FIG. 5D). The quantification of ASI SKN-1::GFP was performed with ImageJ using a sliding paraboloid algorithm for reducing the background followed by edge detection. The t-test was conducted with Welsh correction since the standard deviations were not equal (FIG. 5E). The GSTp::GFP intensity was measured same as ASI SKN-1::GFP but with ×5 magnification. The GSTp::GFP quantification was done using the whole worm signal following the same protocol as intestinal SKN-1::GFP (FIG. 5G).

Locomotion Assays. To measure locomotion, worms were subjected to 30 seconds video recording on a Zeiss Axio Zoom. V16 fluorescence dissecting microscope equipped with Axiocam 503 and ZEN2 software. Bending rate (the number of body-bends-per-seconds) was measured by placing live animals on a plate containing M9 buffer, filming for 30 seconds and counting the number of bends. Healthiness of the worms was measured by their bending rate of young, middle aged and aged animals. For rotenone experiment synchronized N2 and skn-l(zu135) L1 young adult worms were placed on NGM plates with 100 μM hydralazine for three days. Worms were then transferred to a plates containing either 50 μM rotenone plus 100 μM hydralazine, or 50 μM rotenone for 6 h. The results were obtained from 3 individual trials.

### Example 2: Hydralazine Triggers Mitochondrial Biogenesis and Restores Cellular Energetics

Progressive deterioration of mitochondrial function during aging disrupts the cellular energetics homeostasis and triggers the onset of several age-related diseases. Since neurons are primarily post mitotic tissues, energy deprivation (namely ATP) caused by compromised mitochondrial function, significantly affects their cellular homeostasis and contributes to the pathophysiology of various neurodegenerative diseases such as Alzheimer's disease (AD), Parkinson's disease (PD), and Huntington's disease (HD) (2, 4, 5). Therefore, reactivation of mitochondrial function and restoration of the cellular energetics by therapeutic intervention is a highly effective approach to combat diseases like AD and PD (2).

Over the last two decades, several laboratories have tried different strategies to rejuvenate and regenerate the mitochondria by activating critical players such as the sirtuin family of enzymes that use NAD as a cofactor to deacetylate other critical enzymes that control mitochondrial biogenesis, function and cellular energetics (1,2,6,7). As shown hereinabove, hydralazine restores redox cellular homeostasis and protects cells from stress induced death. Thus, the inventors investigated whether hydralazine has any positive impact on cellular energetics as well as mitochondrial biogenesis and function in both control (SH-SY5Y) and disease model cells (tau overexpressed HEK293 cells). It is reported herein, for the first time, that hydralazine can activate several key enzymes that directly control cellular energetics, mitochondrial biogenesis and function.

SH-SY5Y neuroblastoma cells were treated with doxorubicin (0.2 μM), a cytotoxic compound that depletes the NAD pool of the cells and changes morphological features that mimic neurodegeneration such as loss of neuritis (8, 9). These cells were treated with hydralazine (0, 10 and 20 μM) or vehicle for 24 and 48 hours. The result in FIG. 9A clearly show that hydralazine protects cells in a dose-dependent manner against doxorubicin induced cell death as determined by cell viability assay. A consistent pattern of cell rescue from stress induced death was observed in cells treated with hydralazine. Next, the inventors focused on NAD to see if hydralazine also controls cellular energetics via activation of the NAD pathway. Total NAD content was quantified in untreated and hydralazine treated cells using the EnzyChrom NAD/NADH Assay Kit. The results in FIG. 9B show that hydralazine increases NAD level of untreated cell by 50%, and the increase is concentration dependent. Importantly, it also has a significant effect on restoring NAD level in doxorubicin treated cells. Isoniazid, a distant hydralazine homologue, was used as negative control (FIG. 9B).

Since a significant increase in the cellular NAD level was observed as a result of hydralazine treatment in both unstressed and stressed cells, the inventors focused on the activity of enzymes involved in NAD synthesis and enzymes that function in a NAD-dependent manner. The key regulator enzyme, NMNAT-1 (directly involved in NAD biosynthesis), and the NAD-dependent enzymes SIRT1 (cytosolic/nucleus), and SIRT5 (mitochondrial specific) (involved in maintaining cellular energetics) were studied to determine if their levels are up-regulated by hydralazine. Quantitative RT-PCR analysis of the respective transcripts revealed that hydralazine indeed increased the transcription level of these enzymes after treatment for 12 hours (FIG. 10A). Western blot analysis also showed that these enzymes were up-regulated at the protein level (FIG. 10B). All of these data demonstrate that hydralazine is capable of modulating NAD biogenesis and sirtuin pathways both having direct impact on mitochondrial function and biogenesis. Cytochrome c oxidase subunit IV (COX-IV), the critical subunit of the electron transfer chain complex of mitochondria, has been found to be defective in both aging and AD (2, 5). The data in FIG. 10B also shows a significant increase in the level of this enzyme with hydralazine treatment after 48 hours (FIG. 10B). Next, the inventors determined whether hydralazine has any effect on mitochondrial biogenesis and function.

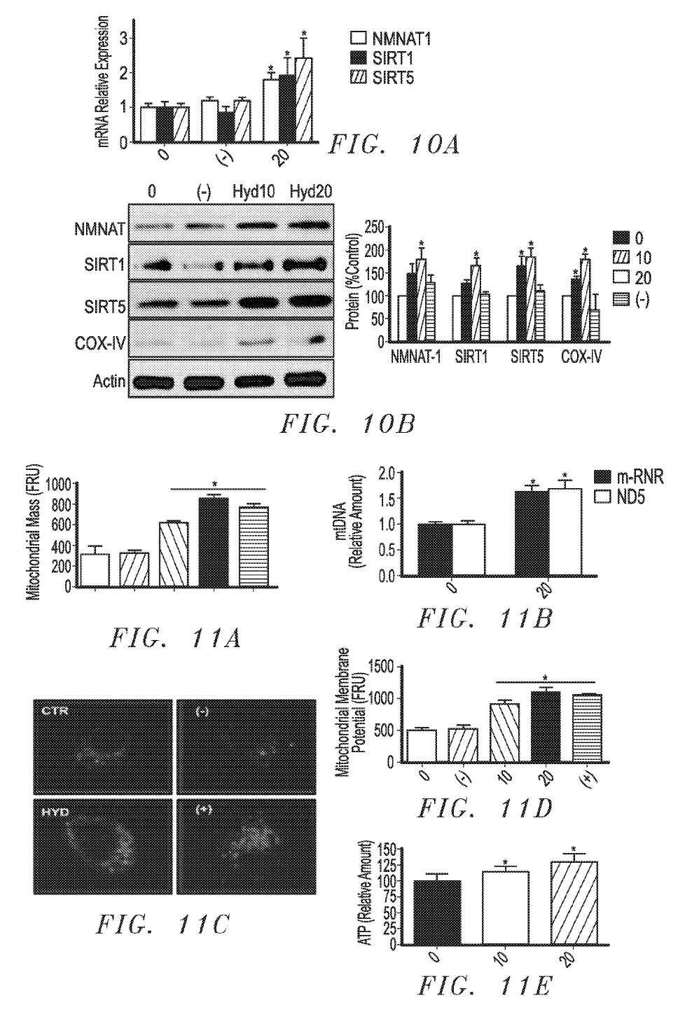

Several parameters directly linked to intact mitochondria itself, namely mitochondrial mass, mtDNA copy number (as representatives of mitochondrial biogenesis), mitochondrial membrane potential (reporter of mitochondrial function) and ATP level as a signature of energetics (FIGS. 11A to 11E) were measured. Using fluorochrome Mito tracker deep red (this probe crosses the mitochondrial membrane and accumulates in active mitochondria), the inventors analyzed mitochondrial mass in hydralazine treated SH-SY5Y cells. There was a sharp increase in mitochondrial mass when cells were treated with increasing concentrations of hydralazine (0, 10 and 20 μM) for 72 hours (FIG. 11A). Isoniazid (20 μM) and resveratrol (20 μM) were used as negative and positive controls respectively). Mitochondrial mass was also evaluated in parallel by measuring the relative amount of mitochondrial to nuclear DNA. Two different set of primers (ND5 and m-RNR) were used to amplify mt-DNA in qPCR reactions (FIG. 11B).

There is a significant increase (more than 50%) in mt-DNA copy number after 72 hours treatment of hydralazine (FIG. 11B), which is strong evidence that hydralazine triggers mitochondrial biogenesis. Next, the impact of hydralazine-induced improvement in mitochondrial function on global cellular energetic state was evaluated. The mitochondrial membrane potential was measured as an indicator of cellular energetics and ATP content. Confocal microscopic imaging and flow cytometry analysis of mitochondrial membrane potential (ΔΨm) by TMRE (tetramethylrhodamine ethyl ester), a positively charged fluorophore that accumulates inside active mitochondria (10), showed that hydralazine improves mitochondrial function in a dose dependent manner (0, 10 and 20 μM), whereas the hydralazine analog isoniazid (20 μM) did not have a positive effects on mitochondrial function (FIG. 11C-D). As mitochondria are the major site of ATP generation, total ATP content was measured as a reporter of mitochondrial activity in cells treated with hydralazine. The results show that hydralazine elevated ATP level in a concentration dependent manner (0, 10 and 20 μM) (FIG. 11E).

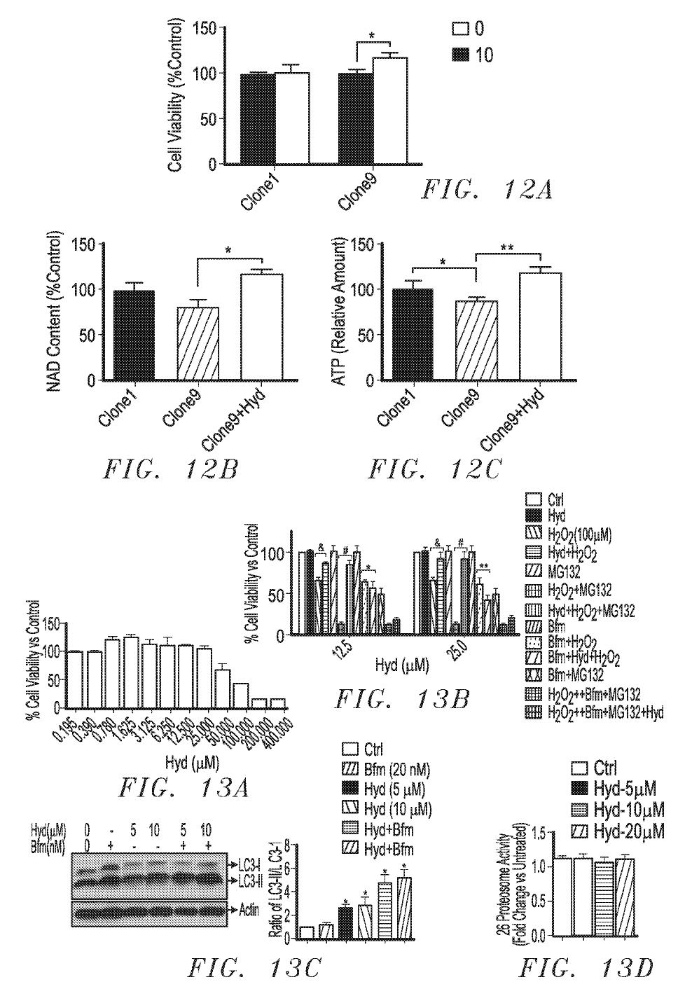

Since energy deprivation and cell growth retardation are often observed in AD, the inventors used a cell-based AD model (using clones of mutant tau described hereinabove) to determine if hydralazine can revert both processes. Two monoclonal cell lines of HEK293 overexpressing mutated tau-RD (repetitive domain; P301L/V337M) were selected; 1) Clone 1: used as control expressing truncated tau from amino acid 244 to 372, with P301L/V337M mutations which produces soluble non-toxic tau, and 2) Clone 9: which generates an aggregated form of tau (11). Three parameters were measured: cell viability, NAD, and ATP content. Based on the results shown in FIGS. 12A-C, it is clear that Clone 9, which carries tau tangles and aggregates, shows improvement in all three aspects.

All these data clearly demonstrate for the first time that hydralazine triggers mitochondrial biogenesis and restore cellular energetics. Most importantly, from a clinical perspective, these data indicate that hydralazine should be tested in an in vivo mouse AD model to determine if these compromised parameters can be reverted to improve physiological and biochemical function, namely mitochondrial function and energetics.

### Example 3: Hydralazine Activates Autophagy and Reduces Proteotoxicity

Several layers of cellular defense mechanisms have evolved to maintain good quality of proteins for normal cellular functions. Restoring normal cellular functions that decline during aging and other pathological conditions such as neurodegenerative diseases, is challenging. Several drugs and experimental interventions have been tested in non-vertebrate and vertebrate disease and aging models to determine if the cellular homeostasis can be restored. Other than calorie restriction (with few exceptions), most of the interventions have failed so far as they cannot modulate multiple master regulatory elements simultaneously to build the crosstalk between or among the relevant pathways. As shown above, hydralazine controls two main pathways namely Nrf2 and SIRT that are directly involved in modulating most of the defense machineries, mitochondrial function and cellular energetics. Most importantly, beneficial effects of hydralazine in AD cell models were demonstrated in terms of activation of Nrf2 and energetics.

There are two distinct degradation machineries in cell, autophagy and proteasome, both found impaired in aging and neurodegeneration. These two are the last layers of cell defense protecting by removing aggregated proteins and damaged organelles to maintain good quality of proteins and organelles. Autophagy is a degradation mechanism involved in maintaining cellular homeostasis by clearing long-lived misfolded proteins and damaged organelle (1). However, misfolded proteins are often found in aggregated form in diseases like AD, PD, and HD where autophagy is also often found to be impaired (2, 3). Next, the inventors examined autophagy and proteasomal degradation to determine if hydralazine plays a role in activating these machineries. The data clearly demonstrate, for the first time, that hydralazine activates the degradation pathways via autophagy and not the proteasomal machinery.

Substantial evidence has now been accumulated to support the hypothesis that proteotoxicity is the causative factor for neuronal cell death in aging and neurodegenerative diseases. For example, in Huntington's disease (HD), which is a hereditary ailment, neurodegeneration is caused by a single-gene mutation. A CAG expansion in the huntingtin (HTT) gene results in production of a mutant HTT with polyglutamine tail (polyQ-HTT), which is associated with the misfolding, aggregate formation and cytotoxicity (4,5). Autophagy can reduce mutant huntingtin protein levels and its toxicity but its impairment at different regulatory steps contributes to the disease progression. Hence, autophagy is becoming an attractive target to treat neurodegenerative disorders through the selective degradation of abnormally folded proteins by the lysosomal pathway (6).

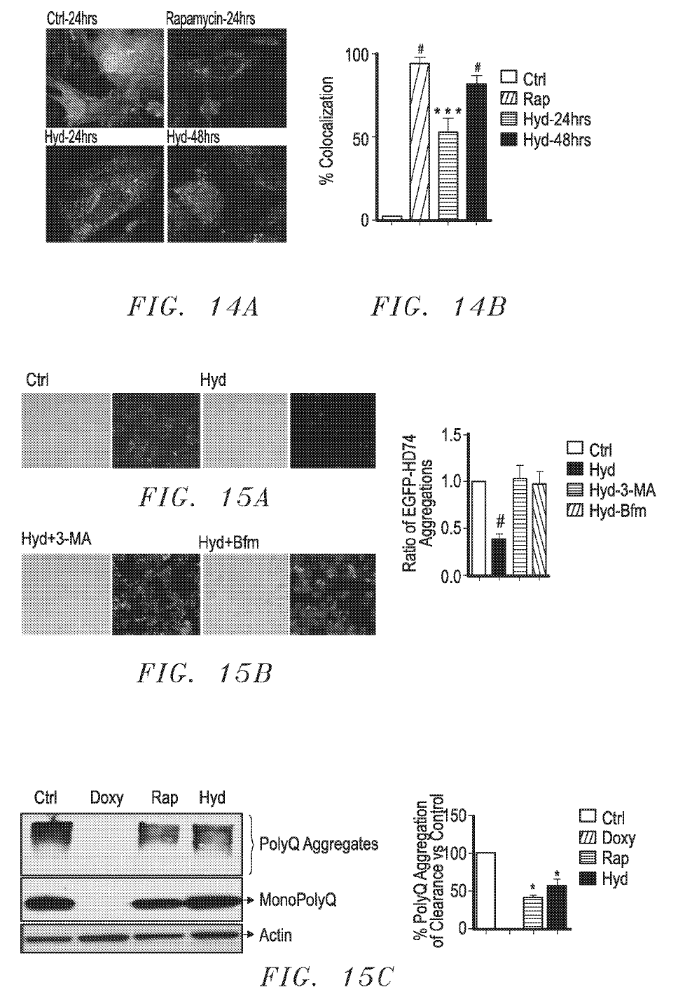

The possibility of hydralazine playing a role in activating degradation systems was investigated. SH-SY5Y cells were treated with different concentrations (0-400 μM) of hydralazine to determine the toxicity level. A cell titer glow luminescence assay was used to measure cell cytotoxicity. As shown in FIG. 13A, no toxicity was observed in SHY-SY5Y cells treated with hydralazine (Hyd) after 48 hours with the concentration up to 25 μM. However, with higher concentrations (50 μM and above) a significant decrease in cell viability was observed compared to control. Hydralazine was used at concentrations less than 25 μM in all subsequent studies. FIG. 13B clearly demonstrates that proteasomal inhibition by MG132 does not have an impact on hydralazine's ability to protect cells against oxidative stress while autophagy inhibition with bafilomycin significantly reduces the ability of hydralazine to protect cells under similar conditions. These results show that hydralazine mediates protection of cells under oxidative stress conditions by activating autophagy. To further confirm the activity of hydralazine, western blot analysis was performed to track autophagy marker LC3 in the presence and absence of bafilomycin. LC3, microtubule-associated protein 1 light chain 3, which is a homologue of Apg8p essential for autophagy in yeast, is processed posttranslationally into LC3-I in cytosol. LC3-I is then converted into LC3-II, which associates with autophagosome membranes. The ratio of LC3-II/LC3-I determines the status of the autophagy process. Based on the data presented FIG. 13C, it is clear that hydralazine induces autophagic flux, which is evident from an increase in the ratio of LC3-II/LC3-I during interference in fusion between autophagosome and lysosome. Proteasomal activity was measured in parallel to determine if hydralazine has any effect on activation of proteasomal function. Based on the data presented in FIG. 13D, it is clear that hydralazine has no significant effect on 20S proteasomal function. All these data together show that hydralazine activates autophagy in dose dependent manner, a role that has not been reported before.

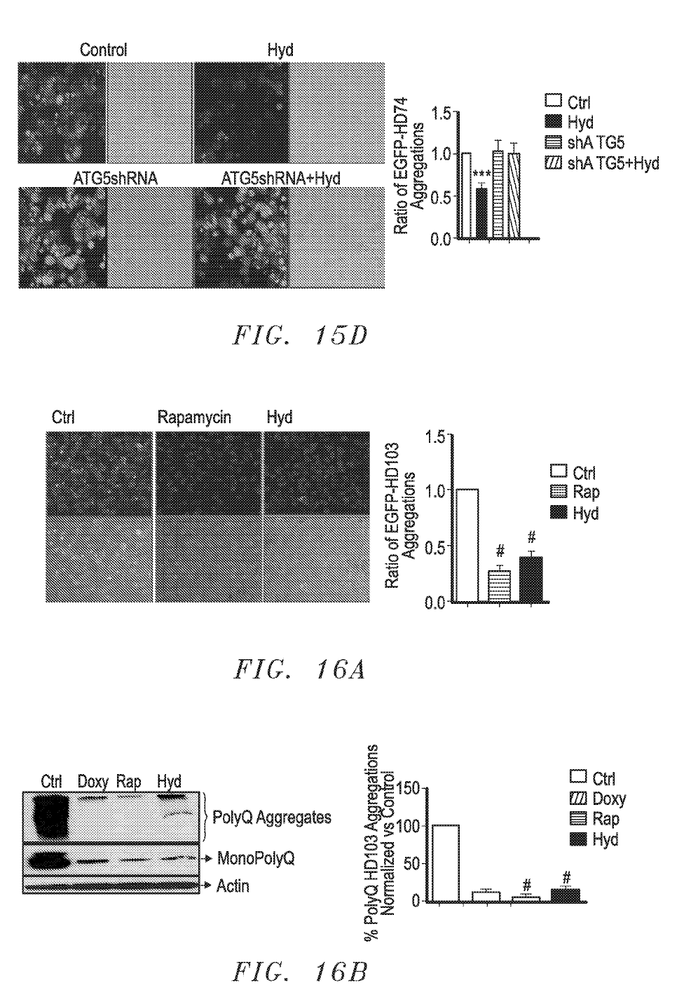

To further validate the present invention autophagic flux using mRFP-GFP tandem fluorescent-tagged LC3 (tfLC3) was measured. tfLC3 protein is one of the most widely used markers for the detection of autophagosomes and autophagolysosomes, two key structures in macroautophagy (8). tfLC3 was knocked-in to monitor autophagic flux in MEF cells based on different pH stabilities of GFP and mRFP (9). In autophagosome environment both mRFP and GFP tags of tfLC3 can fluoresce so autophagosome appear as yellow puncta (green GFP+red mRFP). But after autophagosomal fusion to lysosomes to form the functional autophagolysosome the GFP signal is quenched by the acidic environment of the lysosome and hence autophagolysosomes appear as a red puncta in the image. As shown in FIG. 14, these results demonstrate a time-dependent decrease in the concentration of autophagosomes after hydralazine treatments and an increase in the concentration of autophagolysosomes confirming the induction of autophagic flux in MEF cells by hydralazine. Rapamycin was used as a positive control.

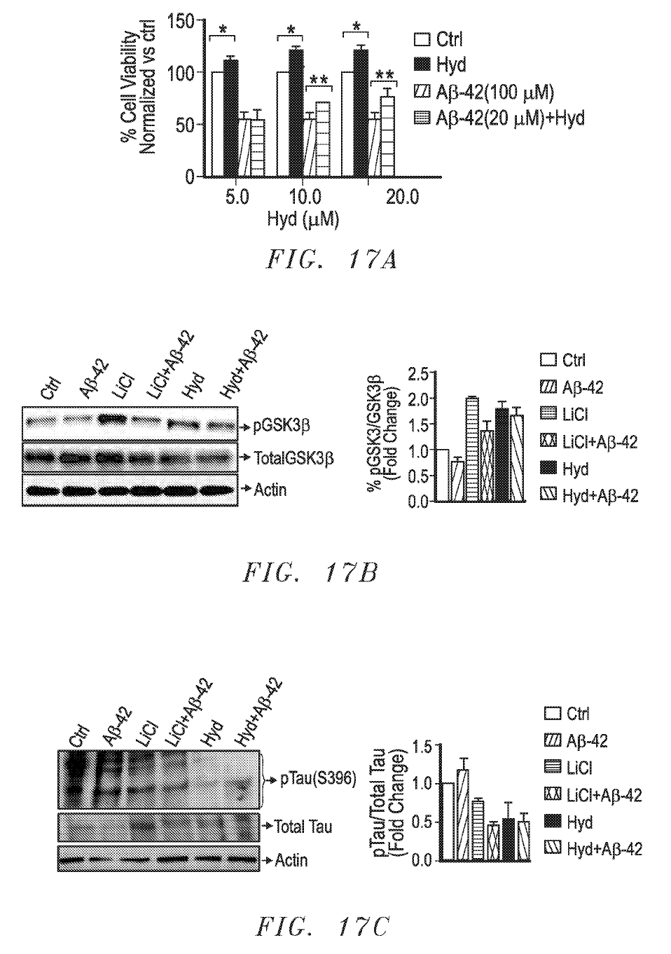

Next, a Huntington's disease cell model was used to show that hydralazine facilitates clearance of mutant Huntington aggregates via autophagy. To investigate the clearance of HDQ74 (74 repeats of poly Q), the inventors used a stable doxycycline-inducible PC12 cell line expressing EGFP-tagged HDQ74 in which transgene expression were induced by doxycycline addition to the cell media and switched off by doxycycline removal. Cells were then incubated with 10 μM hydralazine for 48 hours. Confocal analysis showed that treatment with hydralazine significantly reduced the EGFP-HDQ74 aggregates compared to the control (FIG. 15A). The ability of hydralazine to remove polyQ aggregates was tested in the presence of 3-Methyladenine (3-MA), a known PI3K inhibitor commonly used to inhibit autophagy, and bafilomycin, a known inhibitor of autophagolysosomal flux. Treatment of cells with 3-MA and bafilomycin prior to drug treatment abrogated hydralazine-mediated clearance of Poly Q aggregates as shown by increased percentage of cells with elevated EGFP-HDQ74 signal (FIG. 15B). This result further confirmed that hydralazine is capable of inducing autophagy. Additional western blot analysis using an antibody against the EGFP tag of mutant huntingtin protein (HDQ74) confirmed that hydralazine treatment leads to an efficient removal of poly Q aggregates (FIG. 15C). Rapamycin, a known autophagy inducer, was used as positive controls. Further, the autophagy-inducing property of hydralazine was confirmed by genetic manipulation. ATG5, one of the critical genes for autophagosome activation, was knocked down to determine if hydralazine can activate alternative degradation machinery to reduce poly Q aggregates. As shown in FIG. 15D, hydralazine treatment failed to clear EGFP-HDQ74 aggregates in autophagy protein 5 (ATG5) knock-down cells, which further proves that hydralazine activates the autophagy process selectively to remove aberrant higher-order aggregates.

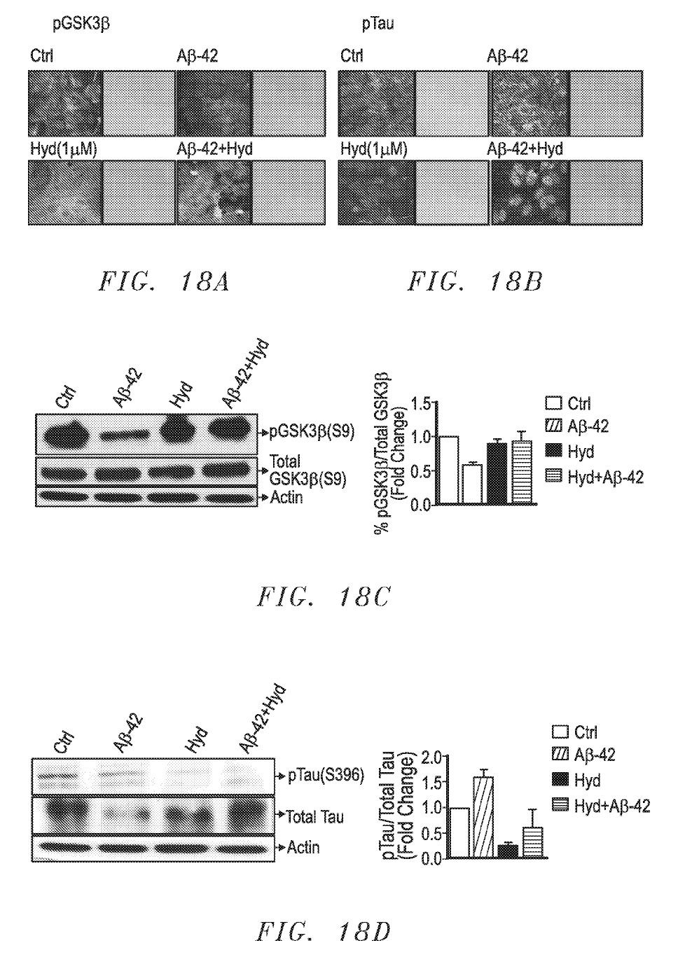

Another Huntington cell line (HeLa HD103Q) was used to confirm the previous findings by confocal analysis and western blot that autophagy induction by hydralazine is not cell type dependent. As shown in FIGS. 16A and 16B hydralazine also induces autophagy in the Huntington cell line HeLa HD103Q cell line, as is evident from the disappearance of higher order poly Q aggregates.

All these observations from this study demonstrate for the first time that FDA approved anti-hypertension drug hydralazine is a novel autophagy inducer. Mouse models of different neurodegenerative diseases (AD, PD and HD) can be used to determine if the physiological functions (cognitive function, memory) are restored and the proteotoxic elements are cleared in selective regions of brain (cerebral cortex, hippocampus, substantia nigra).

### Example 4: Hydralazine Attenuates Tauopathy in AD Disease Models

Aberrant formation of filamentous structures from aggregated tau leads to extensive loss of neuronal cells in several neurological disorders known as tauopathies manifesting usually as different forms of dementia [3,4]. Tau proteins extracted from the brain of patients with tauopathies are often found to be hyper-phosphorylated. The accepted hypothesis is that the hyper-phosphorylated form of tau losses its ability to interact with the C-terminus of tubulin, the building block of microtubule, which in turn result in destabilization of microtubules and aberrant formation of filamentous aggregates that give rise to neurofibril tangles or NFTs (5). In the last few years, scientists have been trying to understand the mechanism/s regulating tau phosphorylation and its toxicity linked to AD disease. It is now known that glycogen synthase kinase-3β (GSK3), a serine/threonine kinase involved in a plethora of cellular mechanisms such as glycogen synthesis, cell survival, and cell division is also responsible for tau phosphorylation [6]. Several studies have shown that GSK3 activity is significantly higher in AD patients driving the formation of both neurofibrillary tangles and amyloid plaques in AD brain [7, 8, 9]. Therefore, GSK3 inhibition has been considered a relevant strategy for the treatment of Alzheimer's disease [10, 11].

The beneficial effects of hydralazine treatment in disease model cell lines were demonstrated hereinabove. When susceptible AD model cells, overexpressing mutant tau RD domain, were treated with hydralazine, cellular homeostasis and mitochondrial function were restored and cells were rescued from stress-induced death. Based on the observations described above, and to specifically test hydralazine protective properties, the inventors used SH-SY5Y neuronal cells to determine if hydralazine can rescue cells from Aβ induced neurotoxicity (Aβ peptide is known to induce tau hyper-phosphorylation). In this use of the present invention, cells were treated with a fixed concentration of Aβ (1-42) peptide (20 μM) and an increasing concentration of hydralazine. After 24 hour treatment, cell viability was measured using the cell titer glow luminescence assay. As shown in FIG. 18A, only 46% of cells treated with Aβ (1-42) peptide survived while in hydralazine treated cells this number was closer to ˜70-80%. Protection of cells from toxic effects of tangled phosphorylated tau by hydralazine cannot be easily explained by the activation of previously described pathways. There is no evidence in the literature that any of previous mechanisms (i.e. autophagy, Nrf2, or mitochondrial activation) is involved in reducing NFT toxicity [1, 2]. So the only possible explanation for acquired protection must lie upstream of NFTs, which in this case is tau phosphorylation. Considering the substantial role GSK3 plays in AD pathology by contributing to the formation of neurofibrillary tangles and amyloid plaques, the inventors hypothesized that hydralazine may function as a GSK3 inhibitor (8, 9).

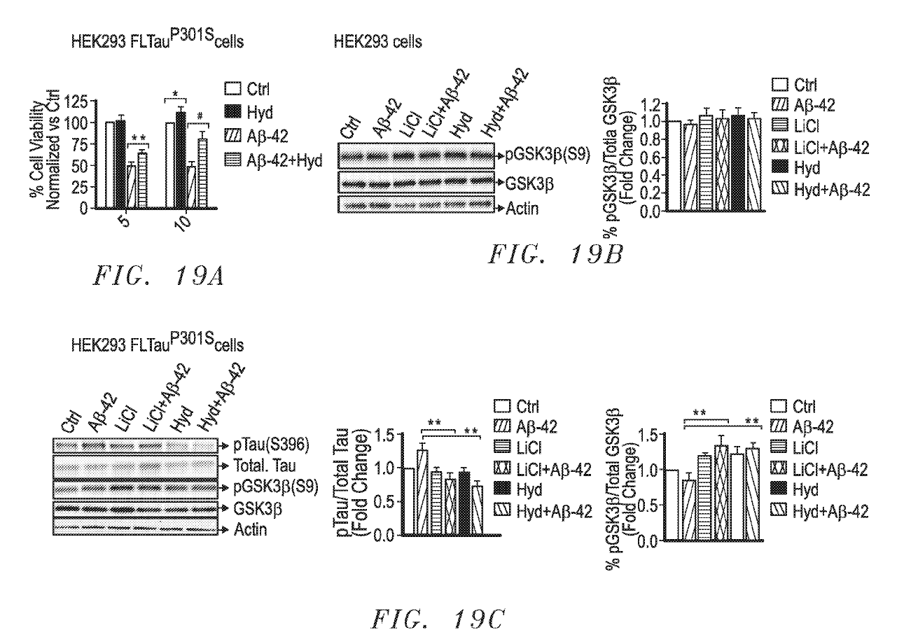

SH-SY5Y cells were treated with hydralazine in the presence and absence of Aβ (1-42) for 24 hours. A known GSK3β inhibitor LiCl (lithium chloride) (10 mM) was used as a positive control [9]. After the treatment, cells were lysed and equal amounts of total lysate were resolved on SDS-PAGE for western blot analysis using phospho-GSK3β (S9) (phosphorylation at this residue reduces the GSK3β kinase activity) and pTau antibodies [12,13]. As shown in FIG. 18B, treatment with 10 μM hydralazine increases inactive phosphorylated form of pGSK3β compared to the control and Aβ (1-42) treated samples. It is also known that hyper-phosphorylation of Tau at S396 causes the formation of NFTs in AD [2,3]. The inventors analyzed the level of Tau phosphorylation at S396 (FIG. 18C). Comparing to the positive control hydralazine treatment reduces the hyper-phosphorylation of Tau even more. All these data (FIGS. 17A-17C) demonstrate that hydralazine protects the neuronal cells from Aβ (1-42) induced toxicity and inhibits GSK3β activity and hyper-phosphorylation of Tau significantly.

Another relevant model of primary neuronal cells, was used to demonstrate the effectiveness of hydralazine. Immunofluorescence assays were performed and the phosphorylation status of GSK3β and Tau proteins were analyzed in primary cells using pGSK3β (S9) and pTau (S396) antibodies. Primary cortical neurons were isolated from the cortex of 0 day pups, cultured for two weeks, and were transfected with tdTomato plasmid to visualize structural and morphological changes of neurons. On Day 21, cells were treated with 1 μM hydralazine in the presence or absence of Aβ (1-42) peptide for 24 hours. At the end of the treatment cells were fixed and immunostained with respective antibodies. As shown in FIGS. 18A-B, treatment of cultured neurons with Aβ in the presence of hydralazine increased the level of inactive phosphorylated form of pGSK3f (FIG. 18A green signal) and decreased the level of pTau (FIG. 18B green signal). These observations were confirmed using Western blot analysis, as shown in FIGS. 18C-D. These analyses confirmed a marked increase in the level of the inactive phosphorylated form of pGSK3β (S9) and a reduced level of pTau with treatment of hydralazine in primary cells stressed by Aβ (1-42) peptide.

Studies with SH-SY5Y and primary neurons show that hydralazine can rescue cells from Aβ (1-42) toxicity by regulating the phosphorylation of GSK3β and dephosphorylation of Tau proteins. The next question answered was whether hydralazine protects cells from amyloid and NFT toxicity known as the main molecular basis for AD pathogenesis. To mimic the cellular environment of AD disease a stable HEK293 cell line (HEKTauP301S) overexpressing mutated full length Tau (P301S) was used. This cell line was challenged with Aβ (1-42) peptide to determine if hydralazine can protects HEKTauP301S cells. The data shown in FIG. 19A confirms that hydralazine protects Tau-overexpressing cells from Aβ (1-42)-induced toxicity. Hydralazine also inhibits Aβ-induced Tau phosphorylation and upregulates the phosphorylation of GSK3β in HEKTauP301S cells (FIG. 19C). The inventors also measured the expression of GSK3β in HEKT293 cells as control but were not able to detect a significant upregulation in phosphorylation level of GSK3β indicating that hydralazine mode of action is stress dependent.

Hydralazine induces neuronal plasticity. Using a global proteomics screening the inventors further found that hydralazine increases the expression of neuropeptide Y by 3-fold, synaptotagmin by ˜2.5 fold, and other neuronal differentiation marker to various extents (data not shown). Since all these proteins play a major role in neurogenesis, differentiation, and plasticity the inventors took a closer look at the role of hydralazine in improving neuronal plasticity and physiological architecture of the neuron. Neuronal plasticity, defined as the brain's ability to form new neural connections, allows the neurons to compensate for injury and disease and to adjust their activities in response to new situations or changes in their environment.

The inventors focused on the neuronal spine, a small membranous protrusion off neuron's dendrite where neurons receive input from a single synapse. Dendritic spines serve as a storage site for synaptic strength and help transmit electrical signals to the neuron's cell body. These spines have a major role in neuroplasticity, the process that allows neurons in the brain to compensate for injury and diseases. Neuronal plasticity was measured by studying the morphological and functional changes of spines and their density in adult primary cortical neurons.

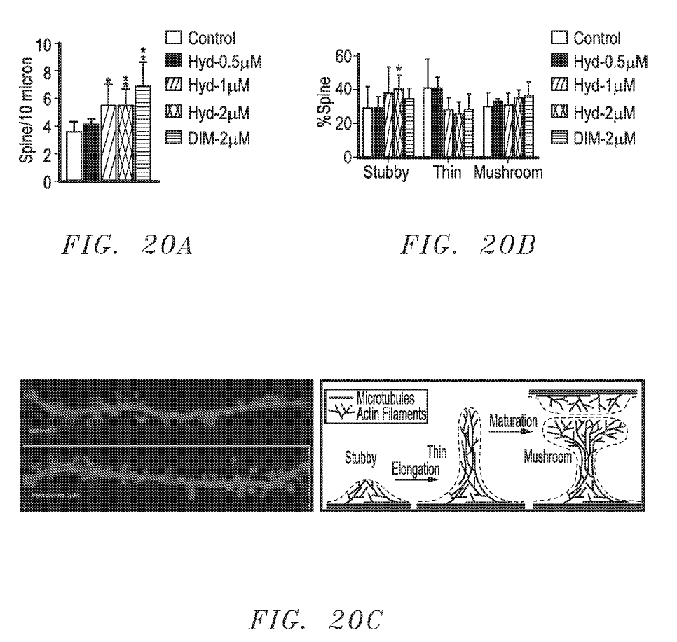

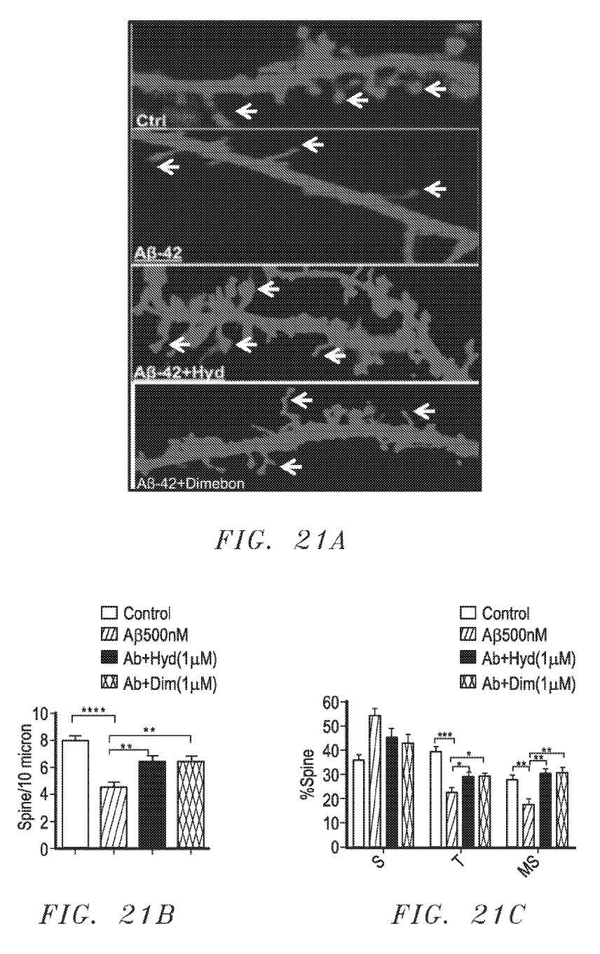

Primary neuronal cells were isolated from the cortex of mice pups on day 0 (P0) and cultured in 24 well plates covered by poly-D-lysine coated coverslips with a density of 1.0×105 for 7 days. Cells were then transfected with tdTomato plasmid and incubated for 14 additional days (4). On day 21, cells were treated with 0.5, 1.0 and 2.0 μM hydralazine. Dimebon (2 μM) was used as a positive control. After 24 hours of treatment, cells were fixed and scanned with a Zeiss microscope at 100× resolution for visualizing spines on the dendrites. The images were analyzed using Neuronstudio software (5). As shown in FIG. 20A, the numbers of spines in a 10 micron length of dendrite was significantly increased with hydralazine treatment in a dose dependent manner compared to the control. Structural formation and the maturation of spines were also analyzed. Treatment with hydralazine increased the stubby formation and numbers of matured spines named mushrooms significantly (FIGS. 20B-20C). These results indicate that hydralazine has the ability to improve neuronal plasticity.

Previous studies have shown that there is a direct correlation between dendritic spine density reduction in hippocampus and memory impairments in mice models of AD. Mushroom spines of excitatory neurons function as “memory spines” that make synapses functionally stronger and are responsible for memory storage. Loss of excitatory mushroom spines hence underlie cognitive decline during AD progression. As shown hereinabove, hydralazine showed a significant effect on increasing spine density and maturation, therefore the inventors next tested the effect of hydralazine on dendritic spine morphology and density altered by Aβ-42 toxicity. Treatment with Aβ-42 oligomers resulted in significant loss of spine density (FIG. 21A, 21B) as a result of a significant reduction in the fraction of thin and mushroom spines (FIG. 21C). Hydralazine treatment resulted in restoration of spine density particularly thin and mushroom lost as a result of Aβ-42 treatment.

Impairment of neuronal plasticity is a common phenomenon observed in neurodegenerative and neuropsychiatric diseases such as Parkinson's disease, Alzheimer's disease, Huntington's disease, and schizophrenia (6-9). In neurodegenerative diseases, neurons lose their connections due to the change in structure and function of the spines, which leads to an impairment of synaptic transmission and memory loss (10). Over the last several years, many laboratories have been conducting research to develop drugs to improve neuronal plasticity and cognitive function (3).

The results from the morphological studies using the present invention demonstrate that hydralazine increases functional synaptic plasticity, which is accompanied by formation of dendritic spines in such a way that more dendritic material is available in synaptic regions where the neurotransmission occurs. In conclusion, these studies demonstrate that hydralazine improves memory and cognitive function that are impaired in neurodegenerative and neuropsychiatric diseases.

As such, it was found that hydralazine activates Nrf2 signaling pathway and protects cells from ROS induced cell death. Hydralazine can activate Nrf2 (nuclear factor erythroid 2-related factor 2), which regulates more than 200 cytoprotective genes that encode proteins and neutralize and detoxify both endogenous and environmental toxins, regulate factors in cell cycle and growth, and facilitate the maintenance of a high quality proteome.

It was further found that hydralazine triggers mitochondrial biogenesis and restores cellular energetics. Hydralazine has been found as a potent activator of two deacetylase enzymes, SIRT1 and SIRT5, which play key roles in various biological processes including mitochondrial biogenesis and restoring mitochondrial function. In fact, the inventors observed these two phenomena with the treatment of hydralazine. As a result, global cellular energetics are elevated as determined by quantification of the ATP level.

Also, hydralazine activates autophagy and reduces proteotoxicity. Hydralazine has the ability to activate specific degradation machinery called autophagy, which is used to remove organelles and protein aggregates (particularly long-lived proteins) in order to reduce proteotoxicity and maintain cellular homeostasis.

The present studies demonstrate the role of hydralazine in attenuating Tauopathy in AD disease cell models. Hydralazine reduces the formation of neurofibrillary tangles (NFTs) (observed frequently in AD patients) by inhibiting the GSK33 kinase and subsequent inhibition of phosphorylation of Tau protein, one of the building block elements of NFTs.

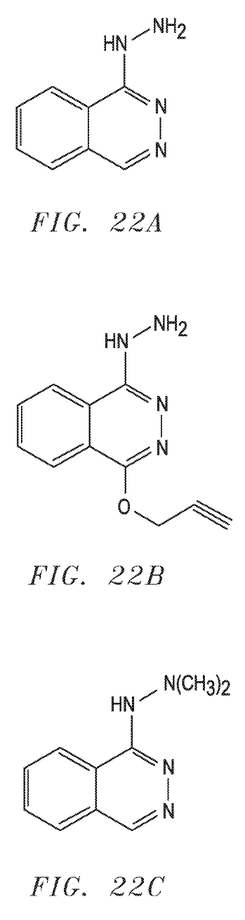

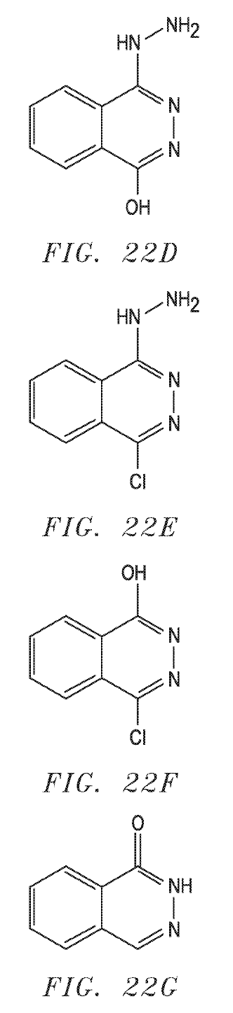

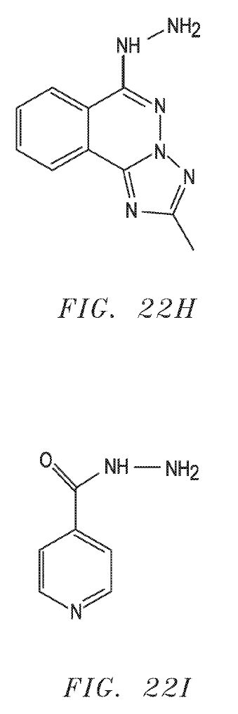

FIGS. 22A to 221 show the structures for the active agents that prevent neurodegeneration or that treat neurodegeneration of neural cells of the present invention. FIG. 22A is hydralazine, FIG. 22B is 1-Hydrazinyl-4-(prop-2-yn-1-yloxy)phthalazine, FIG. 22C is 1,2-Dimethylhydralazine, FIG. 22D is 4-Hydrazinylphthalazin-1-ol, FIG. 22E is 1-Chloro-4-hydrazinylphthalazine, FIG. 22F is 4-Chlorophthalazin-1-ol, FIG. 22G is Phthalazin-1(2H)-one, FIG. 22H is 6-Hydrazinyl-2-methyl-[1,2,4]triazolo[5,1-a]phthalazine, FIG. 22I is Isonicotinohydrazide.

Table 2 shows the activity of the hydralazine and its active analogs identified using the methods taught hereinabove.

| Structure | Name | H₂O₂-EC50 | |
|---|---|---|---|
| 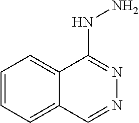 [SMILES: NNc1nncc2ccccc12] | Hydralazine | 6.78 | μM |
| 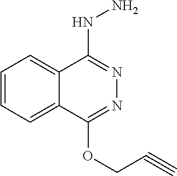 [SMILES: C#CCOc1nnc(NN)c2ccccc12] | 1-Hydrazinyl-4- (prop-2-yn-1-yloxy)- phthalazine | 4.32 | μM |
| 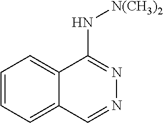 [SMILES: CN(C)Nc1nncc2ccccc12] | 1,2-Dimethylhydralazine | ≥50 | μM |
| 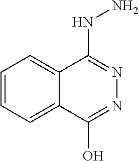 [SMILES: NNc1nnc(O)c2ccccc12] | 4-Hydrazinylphthalazin- 1-ol | ≥10 | μM |
| 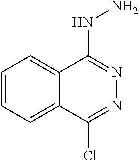 [SMILES: NNc1nnc(Cl)c2ccccc12] | 1-Chloro-4-hydrazinyl- phthalazine | ≥20 | μM |
| 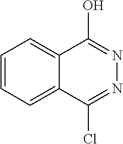 [SMILES: Oc1nnc(Cl)c2ccccc12] | 4-Chlorophthalazin-1-ol | ≥20 | μM |
| 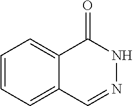 [SMILES: O=c1[nH]ncc2ccccc12] | Phthalazin-1(2H)-one | ≥50 | μM |
| 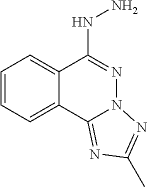 [SMILES: Cc1nc2c3ccccc3c(NN)nn2n1] | 6-Hydrazinyl-2-methyl- [1,2,4]triazolo[5,1-a]- phthalazine | ≥50 | μM |
| 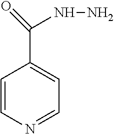 | Isonicotinohydrazide | ≥50 | μM |

Finally, it was found that hydralazine induces neuronal plasticity. Hydralazine has a positive impact on neuronal plasticity as it changes the morphology of spines and their density in adult primary cortical neurons.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### Example 1—References

An, J. H., and Blackwell, T. K. (2003). SKN-1 links C. elegans mesendodermal specification to a conserved oxidative stress response. Genes & development 17, 1882-1893.
Bishop, N. A., and Guarente, L. (2007). Two neurons mediate diet-restriction-induced longevity in C. elegans. Nature 447, 545-549.
Blackwell, T. K., Steinbaugh, M. J., Hourihan, J. M., Ewald, C. Y., and Isik, M. (2015). SKN-1/Nrf, stress responses, and aging in Caenorhabditis elegans. Free radical biology & medicine 88, 290-301.
Bokov, A., Chaudhuri, A., and Richardson, A. (2004). The role of oxidative damage and stress in aging. Mechanisms of ageing and development 125, 811-826.
Brannan, T. S., Maker, H. S., Weiss, C., and Cohen, G. (1980). Regional Distribution of Glutathione Peroxidase in the Adult Rat Brain. Journal of neurochemistry 35, 1013-1014.
Bryan, H. K., Olayanju, A., Goldring, C. E., and Park, B. K. (2013). The Nrf2 cell defence pathway: Keap1-dependent and -independent mechanisms of regulation. Biochemical pharmacology 85, 705-717.
Castillo-Quan, J. I., Li, L., Kinghorn, K. J., Ivanov, D. K., Tain, L. S., Slack, C., Kerr, F., Nespital, T., Thornton, J., Hardy, J., Bjedov, I., and Partridge, L. (2016). Lithium Promotes Longevity through GSK3/NRF2-Dependent Hormesis. Cell reports 15, 638-650.
Clinton, L. K., Blurton-Jones, M., Myczek, K., Trojanowski, J. Q., and LaFerla, F. M. (2010). Synergistic Interactions between Abeta, tau, and alpha-synuclein: acceleration of neuropathology and cognitive decline. The Journal of neuroscience: the official journal of the Society for Neuroscience 30, 7281-7289.
Cook, A. L., Vitale, A. M., Ravishankar, S., Matigian, N., Sutherland, G. T., Shan, J., Sutharsan, R., Perry, C., Silburn, P. A., Mellick, G. D., Whitelaw, M. L., Wells, C. A., Mackay-Sim, A., and Wood, S. A. (2011). NRF2 activation restores disease related metabolic deficiencies in olfactory neurosphere-derived cells from patients with sporadic Parkinson's disease. PloS one 6, e21907.
Dias, V., Junn, E., and Mouradian, M. M. (2013). The role of oxidative stress in Parkinson's disease. Journal of Parkinson's disease 3, 461-491.
Douglas, P. M., and Dillin, A. (2010). Protein homeostasis and aging in neurodegeneration. The Journal of cell biology 190, 719-729.
Edwards, C., Canfield, J., Copes, N., Rehan, M., Lipps, D., and Bradshaw, P. C. (2014). D-beta-hydroxybutyrate extends lifespan in C. elegans. Aging 6, 621-644.
Gao, B., Doan, A., and Hybertson, B. M. (2014). The clinical potential of influencing Nrf2 signaling in degenerative and immunological disorders. Clinical pharmacology: advances and applications 6, 19-34.
Garigan, D., Hsu, A. L., Fraser, A. G., Kamath, R. S., Ahringer, J., and Kenyon, C. (2002). Genetic analysis of tissue aging in Caenorhabditis elegans: a role for heat-shock factor and bacterial proliferation. Genetics 161, 1101-1112.
Hamann, K., Nehrt, G., Ouyang, H., Duerstock, B., and Shi, R. (2008). Hydralazine inhibits compression and acrolein-mediated injuries in ex vivo spinal cord. Journal of neurochemistry 104, 708-718.
Hansen, J. M., Watson, W. H., and Jones, D. P. (2004). Compartmentation of Nrf-2 redox control: regulation of cytoplasmic activation by glutathione and DNA binding by thioredoxin-1. Toxicological sciences: an official journal of the Society of Toxicology 82, 308-317.
Huang, H. C., Nguyen, T., and Pickett, C. B. (2002). Phosphorylation of Nrf2 at Ser-40 by protein kinase C regulates antioxidant response element-mediated transcription. The Journal of biological chemistry 277, 42769-42774.
Itoh, K., Ishii, T., Wakabayashi, N., and Yamamoto, M. (1999). Regulatory mechanisms of cellular response to oxidative stress. Free radical research 31, 319-324.
Kahn, N. W., Rea, S. L., Moyle, S., Kell, A., and Johnson, T. E. (2008). Proteasomal dysfunction activates the transcription factor SKN-1 and produces a selective oxidative-stress response in Caenorhabditis elegans. The Biochemical journal 409, 205-213.
Kaidery, N. A., Banerjee, R., Yang, L., Smirnova, N. A., Hushpulian, D. M., Liby, K. T., Williams, C. R., Yamamoto, M., Kensler, T. W., Ratan, R. R., Sporn, M. B., Beal, M. F., Gazaryan, I. G., and Thomas, B. (2013). Targeting Nrf2-mediated gene transcription by extremely potent synthetic triterpenoids attenuate dopaminergic neurotoxicity in the MPTP mouse model of Parkinson's disease. Antioxidants & redox signaling 18, 139-157.
Kubben, N., Zhang, W., Wang, L., Voss, T. C., Yang, J., Qu, J., Liu, G. H., and Misteli, T. (2016). Repression of the Antioxidant NRF2 Pathway in Premature Aging. Cell 165, 1361-1374.
Kwak, M. K., Wakabayashi, N., Itoh, K., Motohashi, H., Yamamoto, M., and Kensler, T. W. (2003). Modulation of gene expression by cancer chemopreventive dithiolethiones through the Keap1-Nrf2 pathway. Identification of novel gene clusters for cell survival. The Journal of biological chemistry 278, 8135-8145.
Lopes, R. A., Neves, K. B., Tostes, R. C., Montezano, A. C., and Touyz, R. M. (2015). Downregulation of Nuclear Factor Erythroid 2-Related Factor and Associated Antioxidant Genes Contributes to Redox-Sensitive Vascular Dysfunction in Hypertension. Hypertension 66, 1240-1250.
Lu, S. C. (2009). REGULATION OF GLUTATHIONE SYNTHESIS. Molecular aspects of medicine 30, 42-59.
McMahon, M., Itoh, K., Yamamoto, M., Chanas, S. A., Henderson, C. J., McLellan, L. I., Wolf, C. R., Cavin, C., and Hayes, J. D. (2001). The Cap‘n’Collar basic leucine zipper transcription factor Nrf2 (NF-E2 p45-related factor 2) controls both constitutive and inducible expression of intestinal detoxification and glutathione biosynthetic enzymes. Cancer research 61, 3299-3307.
McMahon, M., Thomas, N., Itoh, K., Yamamoto, M., and Hayes, J. D. (2006). Dimerization of substrate adaptors can facilitate cullin-mediated ubiquitylation of proteins by a “tethering” mechanism: a two-site interaction model for the Nrf2-Keap1 complex. The Journal of biological chemistry 281, 24756-24768.
Ong, S. E., Blagoev, B., Kratchmarova, I., Kristensen, D. B., Steen, H., Pandey, A., and Mann, M. (2002). Stable isotope labeling by amino acids in cell culture, SILAC, as a simple and accurate approach to expression proteomics. Molecular & cellular proteomics: MCP 1, 376-386.
Pardridge, W. M. (2002). Targeting neurotherapeutic agents through the blood-brain barrier. Archives of neurology 59, 35-40.
Park, J., Zheng, L., Marquis, A., Walls, M., Duerstock, B., Pond, A., Alvarez, S. V., He, W., Ouyang, Z., and Shi, R. (2014). Neuroprotective role of hydralazine in rat spinal cord injury-attenuation of acrolein-mediated damage. Journal of neurochemistry 129, 339-349.
Park, S. K., Tedesco, P. M., and Johnson, T. E. (2009). Oxidative stress and longevity in Caenorhabditis elegans as mediated by SKN-1. Aging cell 8, 258-269.
Perez, V. I., Cortez, L. A., Lew, C. M., Rodriguez, M., Webb, C. R., Van Remmen, H., Chaudhuri, A., Qi, W., Lee, S., Bokov, A., Fok, W., Jones, D., Richardson, A., Yodoi, J., Zhang, Y., Tominaga, K., Hubbard, G. B., and Ikeno, Y. (2011). Thioredoxin 1 overexpression extends mainly the earlier part of life span in mice. The journals of gerontology Series A, Biological sciences and medical sciences 66, 1286-1299.
Radad, K., Gille, G., and Rausch, W. D. (2008). Dopaminergic neurons are preferentially sensitive to long-term rotenone toxicity in primary cell culture. Toxicology in vitro: an international journal published in association with BIBRA 22, 68-74.
Ramsey, C. P., Glass, C. A., Montgomery, M. B., Lindl, K. A., Ritson, G. P., Chia, L. A., Hamilton, R. L., Chu, C. T., and Jordan-Sciutto, K. L. (2007). Expression of Nrf2 in neurodegenerative diseases. Journal of neuropathology and experimental neurology 66, 75-85.
Rojo, A. I., Innamorato, N. G., Martin-Moreno, A. M., De Ceballos, M. L., Yamamoto, M., and Cuadrado, A. (2010). Nrf2 regulates microglial dynamics and neuroinflammation in experimental Parkinson's disease. Glia 58, 588-598.
Sanders, D. W., Kaufman, S. K., DeVos, S. L., Sharma, A. M., Mirbaha, H., Li, A., Barker, S. J., Foley, A., Thorpe, J. R., Serpell, L. C., Miller, T. M., Grinberg, L. T., Seeley, W. W., and Diamond, M. I. (2014). Distinct tau prion strains propagate in cells and mice and define different tauopathies. Neuron 82, 1271-1288.
Sarlette, A., Krampfl, K., Grothe, C., Neuhoff, N., Dengler, R., and Petri, S. (2008). Nuclear erythroid 2-related factor 2-antioxidative response element signaling pathway in motor cortex and spinal cord in amyotrophic lateral sclerosis. Journal of neuropathology and experimental neurology 67, 1055-1062.
Sheehan, D., Meade, G., Foley, V. M., and Dowd, C. A. (2001). Structure, function and evolution of glutathione transferases: implications for classification of non-mammalian members of an ancient enzyme superfamily. The Biochemical journal 360, 1-16.
Sherer, T. B., Betarbet, R., Testa, C. M., Seo, B. B., Richardson, J. R., Kim, J. H., Miller, G. W., Yagi, T., Matsuno-Yagi, A., and Greenamyre, J. T. (2003). Mechanism of toxicity in rotenone models of Parkinson's disease. The Journal of neuroscience: the official journal of the Society for Neuroscience 23, 10756-10764.
Stadtman, E. R. (1992). Protein oxidation and aging. Science (New York, N.Y.) 257, 1220-1224.
Steele, M. L., and Robinson, S. R. (2012). Reactive astrocytes give neurons less support: implications for Alzheimer's disease. Neurobiology of aging 33, 423.e421-413.
Thimmulappa, R. K., Mai, K. H., Srisuma, S., Kensler, T. W., Yamamoto, M., and Biswal, S. (2002).
Identification of Nrf2-regulated genes induced by the chemopreventive agent sulforaphane by oligonucleotide microarray. Cancer research 62, 5196-5203.
Tong, K. I., Kobayashi, A., Katsuoka, F., and Yamamoto, M. (2006). Two-site substrate recognition model for the Keap1-Nrf2 system: a hinge and latch mechanism. Biological chemistry 387, 1311-1320.
Uno, M., and Nishida, E. (2016). Lifespan-regulating genes in C. elegans. Npj Aging And Mechanisms Of Disease 2, 16010.
Von Otter, M., Landgren, S., Nilsson, S., Celojevic, D., Bergstrom, P., Hakansson, A., Nissbrandt, H., Drozdzik, M., Bialecka, M., Kurzawski, M., Blennow, K., Nilsson, M., Hammarsten, O., and Zetterberg, H. (2010a). Association of Nrf2-encoding NFE2L2 haplotypes with Parkinson's disease. BMC medical genetics 11, 36.
Von Otter, M., Landgren, S., Nilsson, S., Zetterberg, M., Celojevic, D., Bergstrom, P., Minthon, L., Bogdanovic, N., Andreasen, N., Gustafson, D. R., Skoog, I., Wallin, A., Tasa, G., Blennow, K., Nilsson, M., Hammarsten, O., and Zetterberg, H. (2010b). Nrf2-encoding NFE2L2 haplotypes influence disease progression but not risk in Alzheimer's disease and age-related cataract. Mechanisms of ageing and development 131, 105-110.

### Example 2—References

1. Gomes, A. P., Price, N. L., Ling, A. J. Y., Moslehi, J. J., Montgomery, M. K., Rajman, L., White, J. P., Teodoro, J. S., Wrann, C. D., Hubbard, B. P., Mercken, E. M., Palmeira, C. M., De Cabo, R., Rolo, A. P., Turner, N., Bell, E. L., Sinclair, D. A. (2013) Declining NAD induces a pseudohypoxic state disrupting nuclear-mitochondrial communication during aging. Cell. 155 (7): 1624-1638.
2. Andreux P A, Houtkooper R H, Auwerx J (2013) Pharmacological approaches to restore mitochondrial function. Nat Rev Drug Discov. 12(6):465-483.
3. Lanza I R and Nair K S (2010) Mitochondrial function as a determinant of lifespan. Pflugers Archiv. 459, 277-289.
4. Perez-Pinzon M A, Stetler R A, Fiskum G (2012) Novel mitochondrial targets for neuroprotection. J Cereb Blood Flow Metab. 32, 1362-1376.
5. Petrozzi L, Ricci G, Giglioli N J, Siciliano G, Mancuso M (2007) Mitochondria and neurodegeneration. Biosci Rep. 27(1-3):87-104.
6. Langley B, Sauve A (2013) Sirtuin deacetylases as therapeutic targets in the nervous systems. Neurotherapeutics. 10(4): 605-620.
7. Lagouge M, Argmann C, Gerhart-Hines Z, Meziane H, Lerin C, Daussin F, Messadeq N, Milne J, Lambert P, Elliott P, Geny B, Laakso M, Puigserver P, Auwerx J. (2006) Resveratrol improves mitochondrial function and protects against metabolic disease by activating SIRT1 and PGC 1α. Cell 127, 1109-1122.
8. Wang G, Han T, Nijhawan D, Theodoropoulos P, Naidoo J, Yadavalli S, Mirzaei H, Pieper A A, Ready J M, McKnight S L (2014) P7C3 neuroprotective chemicals function by activating the rate-limiting enzyme in NAD salvage. Cell 158: 1324.
9. Guise S, Braguer D, Carles G, Delacourte A and Briand C (2001) Hyperphosphorylation of tau is mediated by ERK activation during anticancer drug-induced apoptosis in neuroblastoma cells. J. Neurosci. Res., 63: 257-267.
10.Perry S W, Norman J P, Barbieri J, Brown E B, Gelbard H A (2011) Mitochondrial membrane potential probes and the proton gradient: a practical usage guide. BioTechniques. 50(2):98-115.
11. Sanders D W, Kaufman S K, DeVos S L, et al. (2014) Distinct tau prion strains propagate in cells and mice and define different tauopathies. Neuron. 82(6):1271-1288.

### Example 3—References

1. Stefan W. Ryter, Suzanne M. Cloonan, and Augustine M. K. Choi. Autophagy: A Critical Regulator of Cellular Metabolism and Homeostasis. Mol. Cells; 36(1), 2013, p 7-16.
2. Metcalf D J, Garcia-Arencibia M, Hochfeld W E, Rubinsztein D C. Autophagy and misfolded proteins in neurodegeneration. Exp Neurol. November; 238(1), 2012, p 22-8.
3. Ralph A Nixon. The role of autophagy in neurodegenerative disease. Nature medicine Vol. 19, 8, 2013, p-983-997.
4. Aislinn J. Williams and Henry L. Paulson. Polyglutamine Neurodegeneration: Protein Misfolding Revisited. Trends Neurosci. Vol-31(10):2008, p 521-528.
5. Sangjune Kim and Kyong-Tai Kim. Therapeutic Approaches for Inhibition of Protein Aggregation in Huntington's disease. Exp Neurobiol. Vol, 23(1): 2014, p 36-44.
6. David C. Rubinsztein, Patrice Codogno and Beth Levine. Autophagy modulation as a potential therapeutic target for diverse diseases. Nature reviews, drug discovery. Vol. 11, 2012, p 709-730.
7. Kevin J. Barnham, Colin L. Masters1 & Ashley I. Bush. Neurodegenerative diseases and oxidative stress. Nature Reviews Drug Discovery. Vol-3, 2004, p 205-214.
8. Kimura, S., Noda, T. & Yoshimori, T. Dissection of the autophagosome maturation process by a novel reporter protein, tandem fluorescent-tagged LC3. Autophagy 3, (2007). p 452-460.
9. Zhou, C. et al. Monitoring autophagic flux by an improved tandem fluorescenttagged LC3 (mTagRFP-mWasabi-LC3) reveals that high-dose rapamycin impairs autophagic flux in cancer cells. Autophagy 8, (2012). p 1215-1226.

### Example 4—References

1. Diane P. Hanger, Brian H. Anderton and Wendy Noble. Tau phosphorylation: the therapeutic challenge for neurodegenerative disease, Cell press. 2009, p 112-119.
2. Alonso A., Zaidi T., Novak M., Grundke-Iqbal I. and Iqbal K. (2001) Hyper phosphorylation induces self-assembly of tau into tangles of paired helical filaments/straight filaments. Proc. Natl Acad. Sci. USA 98, 6923-6928.
3. Grundke-Iqbal I., Iqbal K., Quinlan M., Tung Y. C., Zaidi M. S. and Wisniewski H. M. (1986) Microtubule-associated protein tau. A component of Alzheimer paired helical filaments. J. Biol. Chem. 261, 6084-6089.
4. Goedert M. and Spillantini M. G. (2001) Tau gene mutations and neurodegeneration. Biochem. Soc. Symp. 67, 59-71.2.
5. Kosik K. S. (1992). Alzheimer's disease: a cell biological perspective. Science, 256, 780-783.
6. Lovestone S., Hartley C. L., Pearce J. and Anderton B. H. (1996) Phosphorylation of tau by glycogen synthase kinase-3 beta in intact mammalian cells: the effects on the organization and stability of microtubules. Neuroscience, 73, 1145-1157.
7. Hong M., Chen D. C. R., Klein P. S. and Lee V. M. Y. (1997) Lithium reduces tau phosphorylation by inhibition of glycogen synthase kinase-3. J. Biol. Chem. 272, 25 326-25 332.
8. Imahori K. and Uchida T. (1997) Physiology and pathology of tau protein kinases in relation to Alzheimer's disease. J Biochem. 121, 179-188.
9. MunozMontano J. R., Moreno F. J., Avila J. and DiazNido J. (1997) Lithium inhibits Alzheimer's disease-like tau protein phosphorylation in neurons. FEBS Lett. 411, 183-188.
10.Hooper C, Killick R, Lovestone S. The GSK3 hypothesis of Alzheimer's disease. J Neurochem. 2008, (6):1433-9.
11.Bhat R. V., Budd Haeberlein S. L. and Avila J. (2004) Glycogen synthase kinase 3: a drug target for CNS therapies. J. Neurochem. 89, 1313-1317.
12.Saltiel A. R., Kahn C. R. (2001). Insulin signalling and the regulation of glucose and lipid metabolism. Nature, 414, 799-806.
13.Lizcano J. M., Alessi D. R. (2002). The insulin signalling pathway. Curr. Biol. 12: 236-238.

### Example 5—References

1. Katharina Niesmann, Dorothee Breuer, Johannes Brockhaus, Gesche Born, Ilka Wolff, Carsten Reissner, Manfred W. Kilimann, Astrid Rohlmann & Markus Missler Dendritic spine formation and synaptic function require neurobeachin. Naturecommunications. 2011, p 1-10.
2. Yu-Chih Lin and Anthony J. Koleske, Mechanisms of Synapse and Dendrite Maintenance and Their Disruption in Psychiatric and Neurodegenerative Disorders. Annu Rev Neurosci. 2010; 33: 349-378.
3. Wendou Yu and Bingwei Lu. Synapses and Dendritic Spines as Pathogenic Targets in Alzheimer's disease. Neural Plasticity. 2012, Vol-8.
4. Min Jiang & Gong Chen. High Ca2+-phosphate transfection efficiency in low-density neuronal cultures. 2006, Nature. Protocols, Vol. 2, 695-700.
5. Alfredo Rodriguez, Douglas B. Ehlenberger, Dara L. Dickstein, Patrick R. Hof, Susan L. Wearne. Automated Three-Dimensional Detection and Shape Classification of Dendritic Spines from Fluorescence Microscopy Images. PLos One. 2008, Vol. 3, p 1-12.
6. Peter Penzes, Michael E Cahill, Kelly A Jones, Jon-Eric VanLeeuwen and Kevin M Woolfrey. Dendritic spine pathology in neuropsychiatric disorders. Nat Neurosci. 2011, 14(3): 285-293.
7. Charles R Gerfen. Indirect-pathway neurons lose their spines in Parkinson disease. 2006, Nat Neurosci, Vol. 9, p 157-159.
8. Myrrhe van Spronsen & Casper C. Hoogenraad. Synapse Pathology in Psychiatric and Neurologic Disease. Curr Neurol Neurosci Rep, 2010, 10:207-214.
9. Peter Penzes, Michael E Cahill, Kelly A Jones, Jon-Eric VanLeeuwen & Kevin M Woolfrey. Dendritic spine pathology in neuropsychiatric disorders. Nat Neurosci. 2011, 3, 285-293.
10.Haruo Kasai, Masahiro Fukuda, Satoshi Watanabe, Akiko Hayashi-Takagi and Jun Noguchi. Structural dynamics of dendritic spines in memory and cognition. Trends in Neurosciences. Vol. 33.3, p121-129.

1. A method of protecting neural cells from degeneration or treating degenerated neural cells comprising:
identifying a neural cell in need of protection or treatment from at least one of: radical oxidative stress, increased mitochondrial biogenesis, decreased intracellular protein aggregation or neurofibrillary tangles, decreased cellular NAD or ATP levels, activation of autophagy, removal of protein aggregates, inhibition of GSK3β or Tau protein phosphorylation, or increased neuronal plasticity or dendrite formation; and
providing the neural cell with a therapeutically effective amount of a hydralazine or active bioequivalent thereof sufficient to protect the neural cells from degeneration or treat the neurodegeneration of the neural cells.

2. The method of claim 1, wherein the step of reducing radical oxidative stress comprises activating Nrf2 signaling to neutralize and detoxify the neural cell cytoplasm from radical oxidative stress.

3. The method of claim 1, wherein the active bioequivalent of hydralazine is selected from at least one of 1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, Phthalazin-1 (2H)-one, 6-Hydrazinyl-2-methyl-[1,2,4]triazolo[5,1-a]phthalazine, Isonicotinohydrazide, or salts thereof, and optionally wherein the hydralazine or active bioequivalent thereof is adapted for oral, intravenous, intramuscular, alveolar, intranasal, peritoneal, subcutaneous, enteral, parenteral, rectal, or topical administration; and optionally further comprises one or more pharmaceutically acceptable excipients.

4. (canceled)

5. The method of claim 1, wherein the hydralazine or active bioequivalent thereof is provided in an amount that at least one of: activates deacetylase enzymes, activates SIRT1, activates SIRT5, increases mitochondrial biogenesis, restores mitochondrial function, or elevates cellular NAD or ATP levels; activates autophagy to remove organelles or protein aggregates; reduces the formation of neurofibrillary tangles (NFTs) by inhibiting the phosphorylation of GSK3β kinase or Tau protein; increases neural plasticity or dendrite production; increases the inactive phosphorylated form of pGSK3β; protects neuronal cells from toxicity induced by at least one of Aβ (1-42), amyloid, or NFT: or increases at least one of functional synaptic plasticity or dendrite formation.

6. (canceled)

7. (canceled)

8. (canceled)

9. (canceled)

10. (canceled)

11. (canceled)

12. The method of claim 1, wherein the hydralazine or active bioequivalent thereof is provided in an amount of 10, 20, 30, 40, 50, 60, 70, 80, 90, 100, 125, 150, 175, 200, 225, 250, 275 or 300 milligrams per day.

13. The method of claim 1, wherein the neural cell degeneration is not acrolein-mediated.

14. The method of claim 1, further comprising the step of determining that the neural cell degeneration is not acrolein-mediated, and then providing the subject with the effective amount of hydralazine or active bioequivalent thereof.

15. The method of claim 1, wherein the subject is a human.

16. The method of claim 1, wherein the disease or condition is selected from at least one of a neurodegenerative disease or disorder selected from at least one of non-viral encephalopathy, Alzheimer's disease, Parkinson's disease, amyotrophic lateral sclerosis, Huntington's disease, a tauopathy, an age-related neurodegenerative disease or disorder, muscular sclerosis, a rare genetic neurodegenerative disease, a disease or disorder involving a microbial infection of the nervous system, poliomyelitis, a physical or ischemic injury of the nervous system, seizure, stroke, trauma, epilepsy, a disease or disorder involves the presence of a chemical neurotoxic agent and/or of an oxidative stress.

17. (canceled)

18. A method of treating a neurodegenerative disease or condition in a subject comprising the step of administering a therapeutically effective amount of at least one of Hydralazine, 1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, Phthalazin-1 (2H)-one, 6-Hydrazinyl-2-methyl-[1,2,4]triazolo[5,1-a]phthalazine, Isonicotinohydrazide, or salts thereof.

19. The method of claim 18, wherein the disease or condition is selected from at least one of a neurodegenerative disease or disorder selected from at least one of non-viral encephalopathy, Alzheimer's disease, Parkinson's disease, amyotrophic lateral sclerosis, Huntington's disease, a tauopathy, an age-related neurodegenerative disease or disorder, muscular sclerosis, a rare genetic neurodegenerative disease, a disease or disorder involving a microbial infection of the nervous system, poliomyelitis, a physical or ischemic injury of the nervous system, seizure, stroke, trauma, epilepsy, a disease or disorder involves the presence of a chemical neurotoxic agent and/or of an oxidative stress.

20. The method of claim 18, wherein the hydralazine or active bioequivalent thereof is provided as a pharmaceutically-acceptable salt is selected from acetate, besylate (benzenesulfonate), benzoate, bicarbonate, bitartrate, bromide, calcium edentate, camphorsulfonate (camsylate), carbonate, chloride, chlorotheophyllinate, citrate, edetate, ethanedisulfonate (edisylate), ethanesulfonate (esylate), fumarate, gluceptate (glucoheptonate), gluconate, glucuronate, glutamate, hexylresorcinate, hydroxynaphthoate, hippurate, iodide, isethionate, lactate, lactobionate, lauryl sulfate (estolate), malate, maleate, mandelate, mesylate, methanesulfonate, methylnitrate, methylsulfate, mucate, naphthoate, napsylate, nitrate, octadecanoate, oleate, oxalate, pamoate, pantothenate, phosphate, polygalacturonate, salicylate, stearate, succinate, sulfate, sulfosalicylate, tannate, tartrate, teoclate, toluene sulfonate (tosylate), and trifluoroacetate.

21. The method of claim 18, wherein the neurodegenerative disease or condition is not acrolein-mediated.

22. The method of claim 18, further comprising the step of determining that the neurodegenerative disease or condition is not acrolein-mediated, and then providing the subject with the effective amount of hydralazine or active bioequivalent thereof.

23. The method of claim 18, wherein the subject is a mammal.

24. A method of treating a subject suffering from a neurodegenerative disorder or condition comprising administering an effective amount of a hydralazine or active bioequivalent thereof, wherein the hydralazine or active bioequivalent thereof is effective to at least one of reduce radical oxidative stress, increase mitochondrial biogenesis, decrease intracellular protein aggregation or neurofibrillary tangles, increase cellular NAD and/or ATP levels, activate autophagy, remove protein aggregates, prevent GSK3β or Tau protein phosphorylation, or increase neuronal plasticity and/or dendrite formation.

25. A method of identifying a hydralazine or active analog for preventing or treating a neurodegenerative disorder, the method comprising:
a) measuring at least one of radical oxidative stress, increased mitochondrial biogenesis, decreased intracellular protein aggregation or neurofibrillary tangles, decreased cellular NAD or ATP levels, activation of autophagy, removal of protein aggregates, prevents GSK3β or Tau protein phosphorylation, or increase neuronal plasticity or dendrite formation from neural tissue or cells suspected of having a neurodegenerative disorder from a set of patients;
b) administering a candidate drug to a first subset of the patients, and a placebo to neural tissue or cells from a second subset of the patients;
c) repeating step a) after the administration of the candidate drug or the placebo; and
d) determining if the candidate drug reduces the neurodegenerative disorder that is statistically significant as compared to any reduction occurring in the second subset of patients, wherein a statistically significant reduction indicates that the candidate drug is useful in treating said disease state.

26. (canceled)

27. (canceled)

28. (canceled)

29. (canceled)

30. (canceled)

31. A composition for treating degenerated neural cells comprising a therapeutically effective amount of at least one of Hydralazine, 1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, Phthalazin-1(2H)-one, 6-Hydrazinyl-2-methyl-[1,2,4]triazolo[5,1-a]phthalazine, Isonicotinohydrazide, or salts thereof, sufficient to reduce at least one of radical oxidative stress, increase mitochondrial biogenesis, decrease intracellular protein aggregation or neurofibrillary tangles, increases NAD and/or ATP levels, activate autophagy, remove protein aggregates, prevent GSK3β or Tau protein phosphorylation, or increase neuronal plasticity or dendrite formation, thereby protecting the neural cells from degeneration.

32. A composition for preventing neural cells degeneration comprising a therapeutically effective amount of at least one of Hydralazine, 1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, Phthalazin-1(2H)-one, 6-Hydrazinyl-2-methyl-[1,2,4]triazolo[5,1-a]phthalazine, Isonicotinohydrazide, or salts thereof, sufficient to reduce at least one of radical oxidative stress, increase mitochondrial biogenesis, decrease intracellular protein aggregation or neurofibrillary tangles, increases NAD and/or ATP levels, activate autophagy, remove protein aggregates, prevent GSK3β or Tau protein phosphorylation, or increase neuronal plasticity or dendrite formation, thereby protecting the neural cells from degeneration.

33. The composition of claim 32, wherein the hydralazine or active bioequivalent thereof is adapted for oral, enteral, parenteral, intravenous, intramuscular, pulmonary, rectal, or subcutaneous administration.

34. The composition of claim 32, wherein the hydralazine or active bioequivalent thereof further comprises one or more pharmaceutically acceptable excipients.

35. A method of treating a condition comprising:
identifying a condition treatable by at least one of:
reducing radical oxidative;
increasing mitochondrial biogenesis;
increasing cellular NAD and/or ATP levels;
activating at least one of autophagy, removal of protein aggregates, or prevents GSK3β, or Tau protein phosphorylation;
increasing neuronal plasticity or dendrite formation; or
treating degenerated neural cells; and
providing an effective amount of at least one of Hydralazine, 1-Hydrazinyl-4-(prop-2-yn-1-yloxy)phthalazine, 1,2-Dimethylhydralazine, 4-Hydrazinylphthalazin-1-ol, 1-Chloro-4-hydrazinylphthalazine, 4-Chlorophthalazin-1-ol, Phthalazin-1(2H)-one, 6-Hydrazinyl-2-methyl-[1,2,4]triazolo[5,1-a]phthalazine, Isonicotinohydrazide, or salts thereof, sufficient to reduce at least one of radical oxidative stress, increase mitochondrial biogenesis, decrease intracellular protein aggregation or neurofibrillary tangles, increases NAD and/or ATP levels, activate autophagy, remove protein aggregates, prevent GSK3β or Tau protein phosphorylation, or increase neuronal plasticity or dendrite formation, or degenerated neural cells.
